(19) United States
(12) Patent Application Publication
(10) Pub. No.: US 2026/0198833 A1
(43) Pub. Date: Jul. 16, 2026

(54) PRESSURE-SENSITIVE ELECTROCHEMILUMINESCENT SYNAPTIC DEVICE FOR ELECTROCARDIOGRAM PATTERN CLASSIFICATION AND VISUALIZATION

(71) Applicant: UIF (University Industry Foundation), Yonsei University, Seoul (KR)

(72) Inventors: Cheol Min PARK, Seoul (KR); Woo Joong KIM, Seoul (KR); Gwan Ho KIM, Seoul (KR)

(73) Assignee: UIF (University Industry Foundation), Yonsei University, Seoul (KR)

(21) Appl. No.: 19/226,750

(22) Filed: Jun. 3, 2025

(30) Foreign Application Priority Data
Jan. 15, 2025 (KR) 10-2025-0005930

(51) Int. Cl. A61B 5/318; A61B 5/266; A61B 5/268

(52) U.S. Cl. CPC A61B 5/318; A61B 5/266; A61B 5/268

(57) ABSTRACT

Disclosed is an electrocardiogram (ECG) pattern classification and visualization device including a pressure sensitive electrochemiluminescent synaptic element for classifying and visualizing an electrocardiogram pattern. The electrocardiogram (ECG) pattern classification and visualization device includes: a sensor for measuring an electrocardiogram signal of a living body; an artificial neural network configured to receive the electrocardiogram signal from the sensor as an input and transmit an output via signal propagation; and a synaptic element configured to receive a signal processed by the artificial neural network therefrom and visualize an electrocardiogram pattern based on the received signal.

### CROSS-REFERENCE TO RELATED APPLICATION

This application claims priority from Korean Patent Application No. 10-2025-0005930 filed on Jan. 15, 2025 in the Korean Intellectual Property Office, and all the benefits accruing therefrom under 35 U.S.C. 119, the contents of which in its entirety are herein incorporated by reference.

### BACKGROUND

### Field

The present disclosure relates to a pressure sensitive electrochemiluminescent synaptic element for classifying and visualizing an electrocardiogram pattern. Specifically, the present disclosure relates to an electrocardiogram pattern classification and visualization device including the pressure sensitive electrochemiluminescent synaptic element for classifying and visualizing an electrocardiogram pattern, and an electrocardiogram diagnosis method using the electrocardiogram pattern classification and visualization device.

### Description of Related Art

The integration of displays into artificial synapses may create new types of neuromorphic devices called visual synapses. They combine the functionality of artificial synapses with the ability to visualize their activity in real time. Visual synapses provide direct and intuitive visualization of the activity of artificial synapses, providing several benefits that may advance neuromorphic computing applications. Visual synapses enable real-time monitoring of the activity and changes in artificial synapses. This capability enables scholars to observe and analyze the behavior of synapses as they process information, adapt, and learn. Moreover, the development of motion monitoring systems that enable direct communication at human-device interfaces is enhanced by visual synapses, which help understand and interpret human movements in real time. For instance, in a monitoring system that involves the motion of human bodies, including arms, fingers, and the heartbeat, the physical distance between an artificial synapse and an external communication device varies significantly from a few millimeters to centimeters, requiring lengthy electrical interconnects for long-distance communication. Visual synapses may mitigate these problems, making communication faster with low-power consumption. Numerous examples of visual feedback are found in applications for monitoring human protection and biomedical fields, such as assisting patients using visualized alarms to prevent an acute arrhythmia and facilitating rehabilitation processes.

Synapses capable of simultaneously sensing and learning from a wide range of stimuli, such as pressure, light, temperature, humidity, and magnetic fields have been demonstrated. These devices may be combined with various chromic and self-emitting materials that change their optical properties in response to electrical or chemical signals arising from artificial synapses, enabling the direct, real-time visualization of synaptic activity. In particular, visualization of an artificial synapse associated with various tactile stimuli, such as repetitive tapping and stretching, is of great importance for real-time motion tracking and revising potentially providing an efficient route for physical rehabilitation. Various tactile visual synapses, comprising individual sensors, artificial synapses, and light-emitting elements, have been reported. In some cases, they are physically interconnected with microprocessor units for facile signal conversion of those from the sensor and synapse into the display. However, these bulky tactile visual synapses are rarely suitable for real-time motion monitoring and healthcare systems owing to their poor wearability on the body and high-power consumption. Therefore, the development of a single-device tactile visual synapse is required, where tactile synaptic activity acquired by the device is directly visualized from the device. A single-device tactile visual synapse with full visible color operation may further facilitate feedback-based diagnosis and rehabilitation with high accuracy.

Inspired by a bio-luminescent marine snail, Hinea brasiliana, which emits chemiluminescent light whose intensity increases with the number of taps by a predator on the shell surface of the snail the inventors of the present disclosure present a compact, low-power, full-color, single-device electrochemiluminescent tactile visual synaptic element (ECL-TVS). The proposed ECL-TVS is based on a three-terminal top-gate bottom contact organic electrochemical transistor (OECT), comprising an elastomeric top gate as a tactile receptor, electrochemiluminescent ion-gel (ECL-IG) as a light emitting layer, and a polymeric semiconductor layer as a synaptic channel. While tactile motions imposed to the top gate are represented by the synaptic current of the semiconducting channel as a function of the degree and frequency of the tactile motions, the synaptic activity is simultaneously visualized in the ECL-IG. The three representative red, green, and blue ECL-IGs are employed with different oxidation potentials, enabling the input potential-dependent full-color visualization of the synaptic activity. An energy-efficient (about −3 V, about 34 μW) wearable ECL-TVS panel capable of monitoring subtle distal motions of fingers is demonstrated in which the repetitive finger flexion (and extension) motions are monitored and visually guided using the present thin wearable ECL-TVS in combination with the electrical and optical output feedback algorithm. Furthermore, various types of arrhythmias, which are unwanted irregular heartbeats, are visually monitored and interpreted in real time using a spiking neural network (SNN). The ability to monitor and visualize repetitive finger movements and heartbeat is essential in the rehabilitation and healthcare fields, highlighting the convenience and effectiveness of the present ECL-TVS as a platform for a personalized healthcare system. The inventors of the present disclosure validate its utility as a tool for in-situ monitoring by observing functional enhancements in users' finger movements and the high accuracy of ECG data analysis facilitated by the bioinspired full-color electrochemiluminescent tactile visual synaptic element (ECL-TVS).

### SUMMARY

A purpose to be achieved by the present disclosure is to provide a method for more quickly evaluating a heart health condition through a technology capable of efficiently analyzing and intuitively visualizing an electrocardiogram signal. Thus, not only medical staff but also general users may easily understand and utilize electrocardiogram data, thereby contributing to personalized health management based on the electrocardiogram data. In addition, this technology reduces analysis delay or data interpretation difficulty that may occur in the existing system, and presents a direction in the development of a device that enables simple diagnosis and real-time monitoring. This technology has the potential to increase the convenience in the process of analyzing and visualizing electrocardiogram data and further contribute to expanding its utilization in the medical field.

Purposes according to the present disclosure are not limited to the above-mentioned purpose. Other purposes and advantages according to the present disclosure that are not mentioned may be understood based on following descriptions, and may be more clearly understood based on embodiments according to the present disclosure. Further, it will be easily understood that the purposes and advantages according to the present disclosure may be realized using means shown in the claims or combinations thereof.

One aspect of the present disclosure provides an electrocardiogram (ECG) pattern classification and visualization device comprising: a sensor for measuring an electrocardiogram signal of a living body; an artificial neural network configured to receive the electrocardiogram signal from the sensor as an input and transmit an output via signal propagation; and a synaptic element configured to receive a signal processed by the artificial neural network therefrom and visualize an electrocardiogram pattern based on the received signal.

In accordance with some embodiments of the ECG pattern classification and visualization device, the synaptic element includes: a source electrode; a drain electrode horizontally spaced apart from the source electrode; an organic semiconductor layer deposited on both the source electrode and the drain electrode so as to be in contact with both the source electrode and the drain electrode; an ion-gel layer formed on the organic semiconductor layer; and a gate electrode formed on the ion-gel layer and formed in a shape in which at least one portion of a lower surface thereof is convex downwardly.

In accordance with some embodiments of the ECG pattern classification and visualization device, the synaptic element is configured to receive the processed signal from the artificial neural network via the gate electrode.

In accordance with some embodiments of the ECG pattern classification and visualization device, the ion-gel layer includes: a polymer matrix; an ionic liquid dispersed in the polymer matrix and having electrical conductivity; one or more luminophores dispersed in the polymer matrix and generating electrochemical luminescence in response to electrical stimulation; and a co-reactant transferring electrons to the luminophores in response to the electrical stimulation.

In accordance with some embodiments of the ECG pattern classification and visualization device, the artificial neural network includes a spiking neural network (SNN).

In accordance with some embodiments of the ECG pattern classification and visualization device, the organic semiconductor layer includes poly(3-hexylthiophene) (P3HT).

In accordance with some embodiments of the ECG pattern classification and visualization device, the gate electrode GE is formed in a partial hemispherical shape protruding downwardly.

In accordance with some embodiments of the ECG pattern classification and visualization device, the polymer matrix includes P(VDF-HFP) (poly(vinylidene fluoride-hexafluoropropylene)).

In accordance with some embodiments of the ECG pattern classification and visualization device, the ionic liquid includes 1-butyl-1-methylpyrrolidinium bis(trifluoromethylsulfonyl)imide ([PYR₁₄][TFSI]).

In accordance with some embodiments of the ECG pattern classification and visualization device, the luminophore includes at least one selected from a group consisting of Ru(bpy)₃Cl₂, Ir(dfppy)₂(bpy)PF₆, and Ir(diFppy)₂(pic) (Firpic).

In accordance with some embodiments of the ECG pattern classification and visualization device, the co-reactant may include tripropylamine (TPrA).

In accordance with some embodiments of the ECG pattern classification and visualization device, the ECG pattern classification and visualization device is configured to classify the ECG pattern into N” (Normal), “S” (Supraventricular Arrhythmia), “V” (Ventricular Arrhythmia), “F” (Fusion Beat), and “Q” (Unknown or Noisy Beats) patterns.

In accordance with some embodiments of the ECG pattern classification and visualization device, the synaptic element is configured to visualize different electrocardiogram patterns using different colors.

Another aspect of the present disclosure provides a method for diagnosing electrocardiogram, the method comprising analyzing an electrocardiogram of a living body using the electrocardiogram pattern classification and visualization device as described above.

Tactile visual synapses combine the functionality of tactile artificial synapses with the ability to visualize their activity in real time and provide a direct and intuitive visualization of the activity, offering an efficient route for in-situ health monitoring. Herein, the inventors of the present disclosure present a tactile visual synapse that enables in-situ monitoring of finger rehabilitation and electrocardiograms (ECGs) analysis. Repetitive finger flexion and various arrhythmias are monitored and visually guided using the developed tactile visual synapse combined with an electrical and optical output feedback algorithm. The tactile visual synapse has the structure of an electrochemical transistor comprising an elastomeric top gate as a tactile receptor and an electrochemiluminescent ion-gel as a light-emitting layer stacked on a polymeric semiconductor layer, forming an electrical synaptic channel between source and drain electrodes. The low-power (about 34 W) visualization of the tactile synaptic activity associated with the repetitive motions of fingers and heartbeats enables the development of a convenient and efficient personalized healthcare system.

The effect of the present disclosure includes providing an opportunity to more intuitively grasp a heart health state by implementing analysis and visualization of the electrocardiogram signal using a single device. Thus, medical staff may use the present device for rapid diagnosis and treatment plan establishment, and general users may continuously monitor their health status through a simple interface using the present device. In addition, the present device may reduce the burden of using an existing complex analysis process or expensive equipment, such that the possibility that the present device may be used in a wider environment may be enhanced. In particular, when the present device is integrated with wearable devices based on the characteristics of high portability and energy efficiency of the present device, there is room for contributing to real-time health care and preventive measures.

Effects of the present disclosure are not limited to the effects mentioned above, and other effects not mentioned will be clearly understood by those skilled in the art from the description as set forth below.

In addition to the above effects, specific effects of the present disclosure are described together while describing specific details for carrying out the present disclosure.

### BRIEF DESCRIPTION OF DRAWINGS

The patent or application file contains a least one drawing executed in color. Copies of this patent or patent application publication with color drawing(s) will be provided by the Office upon request and payment of the necessary fee.

FIG. 1A. Bioinspired full-color electrochemiluminescent tactile visual synaptic element (ECL-TVS). (Upper diagrams) Schematic of the sea snails' biological tactile perception & bioluminescence system: (i) the tactile sensory receptors, (ii) synapse, (iii) light emitting cell (photocyte) in the epithelial tissue of glowing sea snail and the corresponding ECL-TVS based on ion-gel-gated element. (Lower diagrams) (i) artificial tactile sensors with dome-shaped elastomeric gate electrode and ion-gel dielectric layer disposed thereunder, (ii) the artificial synapse showing synaptic currents between source and drain electrodes, (iii) artificial light emitting cell with ion-gel containing luminophore and co-reactant which generate light through electrochemiluminescence.

FIG. 1B. Bioinspired full-color electrochemiluminescent tactile visual synaptic element (ECL-TVS). Structure of the bioinspired full-color electrochemiluminescent tactile visual synaptic element (ECL-TVS) and the components that constitute an ECL ion-gel (IG).

FIG. 1C. Bioinspired full-color electrochemiluminescent tactile visual synaptic element (ECL-TVS). Photograph of the bioinspired full-color electrochemiluminescent tactile visual synaptic element (ECL-TVS) from the front view and quarter view; front view: gate electrode pressing the ion-gel (scale bar: 2 mm), quarter view: electrochemiluminescent tactile visual synaptic element (ECL-TVS) and the pressure-sensitive gate electrode. The inset is a photograph of full-color light-emitting ion-gel and a schematic of the ITO source/drain electrode.

FIG. 1D. Bioinspired full-color electrochemiluminescent tactile visual synaptic element (ECL-TVS). Normalized R-, G-, and B-ECL-TVS emission spectra.

FIG. 1E. Bioinspired full-color electrochemiluminescent tactile visual synaptic element (ECL-TVS). Electrical transfer and luminance characteristics of the bioinspired full-color electrochemiluminescent tactile visual synaptic element (ECL-TVS) as a function of gate voltage.

FIG. 1F. Bioinspired full-color electrochemiluminescent tactile visual synaptic element (ECL-TVS). The threshold voltages for electrical transistor and emission of the 3 electrochemiluminescent tactile visual synaptic elements (ECL-TVS) with R-, G-, and B-luminophores.

FIG. 2A. Analysis of the operational mechanism in an electrochemiluminescent tactile visual synaptic element (ECL-TVS). Schematic illustration of the operational mechanism in an electrochemiluminescent tactile visual synaptic element (ECL-TVS).

FIG. 2B. Analysis of the operational mechanism in an electrochemiluminescent tactile visual synaptic element (ECL-TVS). Schematic illustration of a liquid electrolyte-based in-situ measurement system.

FIG. 2C. Analysis of the operational mechanism in an electrochemiluminescent tactile visual synaptic element (ECL-TVS). Schematic illustration of a solid electrolyte-based in-situ measurement system.

FIG. 2D. Analysis of the operational mechanism in an electrochemiluminescent tactile visual synaptic element (ECL-TVS). Absorbance plots of the P3HT layer as a function of doping potential.

FIG. 2E. Analysis of the operational mechanism in an electrochemiluminescent tactile visual synaptic element (ECL-TVS). Plots of transient gate current of the P3HT film with different applied doping potentials.

FIG. 2F. Analysis of the operational mechanism in an electrochemiluminescent tactile visual synaptic element (ECL-TVS). Calculated hole concentration from FIG. 2E.

FIG. 2G. Analysis of the operational mechanism in an electrochemiluminescent tactile visual synaptic element (ECL-TVS). (Upper graph) Real-time plots of PSC under the gate bias of −3.5 V in solid electrolyte-based electrochemiluminescent tactile visual synaptic element (ECL-TVS). (Lower graph) Real-time plots of ECL photocurrent under the gate bias of −3.5 V in solid electrolyte-based electrochemiluminescent tactile visual synaptic element (ECL-TVS). The rise time of PSC (rPSC) and ECL (iECL) represents the timespan between 10% and 90% of the maximum value, while the ECL delay time (Aidelay) is the delay from “Vgs application” to “ECL emission”.

FIG. 2H. Analysis of the operational mechanism in an electrochemiluminescent tactile visual synaptic element (ECL-TVS). Plot of rise time of PSC.

FIG. 2I. Analysis of the operational mechanism in an electrochemiluminescent tactile visual synaptic element (ECL-TVS). Plot of delay time.

FIG. 2J. Analysis of the operational mechanism in an electrochemiluminescent tactile visual synaptic element (ECL-TVS). Plot of the rise time of ECL as a function of applied gate voltage.

FIG. 3A. Visualization of the synaptic activity in an electrochemiluminescent tactile visual synaptic element (ECL-TVS). Plot of the PSC response and the brightness of an R-ECL-TVS with 20 times tactile pulses under negative gate voltage are continuously applied (Vgs=−3.5 V, Vds=−0.1 V, pressure pulse duration=0.1 s, and pressure pulse interval=1 s).

FIG. 3B. Visualization of the synaptic activity in an electrochemiluminescent tactile visual synaptic element (ECL-TVS). Plot of the light intensity and light emitting area of R-ECL-TVS from FIG. 3A with inset photographs displaying the electrochemiluminescence at the 1st, 4th, 8th, 12th, 16th, and 20th tactile pulses. Scale bar: 5 mm.

FIG. 3C. Visualization of the synaptic activity in an electrochemiluminescent tactile visual synaptic element (ECL-TVS). Plot of the PSC responses with different frequencies of pressure pulses.

FIG. 3D. Visualization of the synaptic activity in an electrochemiluminescent tactile visual synaptic element (ECL-TVS). Light intensity at 1st, 2nd, and 10th pulse based on the different pressure frequencies of 0.1 Hz, 0.5 Hz, and 2 Hz.

FIG. 3E. Visualization of the synaptic activity in an electrochemiluminescent tactile visual synaptic element (ECL-TVS). Light emission area at 1st, 2nd, and 10th pulse based on the different pressure frequencies of 0.1 Hz, 0.5 Hz, and 2 Hz.

FIG. 3F. Visualization of the synaptic activity in an electrochemiluminescent tactile visual synaptic element (ECL-TVS). Schematic illustration of the electric field by dome-shape gate electrode and the light emission of the bioinspired full-color electrochemiluminescent tactile visual synaptic element (ECL-TVS).

FIG. 3G. Visualization of the synaptic activity in an electrochemiluminescent tactile visual synaptic element (ECL-TVS). Finite element analysis (FEA) simulation of the electric field distribution in ECL-TVS with a dome-shape electrode contact.

FIG. 3H. Visualization of the synaptic activity in an electrochemiluminescent tactile visual synaptic element (ECL-TVS). Plot of capacitance as a function of ion-gel thickness when the lateral dimension of ion-gels were 2 mm and 5 mm, respectively.

FIG. 4A. Full-color electrochemiluminescent tactile visual synaptic element (ECL-TVS). A plot of the PSC response of an R-electrochemiluminescent tactile visual synaptic element (ECL-TVS), utilizing 20 V_{gs} pulses sequentially increasing in magnitude. The inset photographs display the light emission of R-ECL-TVS at the increasing number of V_{gs} pulses. scale bar: 5 mm.

FIG. 4B. Full-color electrochemiluminescent tactile visual synaptic element (ECL-TVS). Photographs of R, G, and B-ECL-TVS with continuous 15 gate voltage pulses, sequentially increasing in magnitude from −2.2 V to −5 V. Scale bar: 3 mm.

FIG. 4C. Full-color electrochemiluminescent tactile visual synaptic element (ECL-TVS). Plot of the PSC response of integrated R-, G-, and B-electrochemiluminescent tactile visual synaptic element (ECL-TVS), utilising 15 gate voltage pulses comprising three sets of five voltage pulses with different voltage magnitudes (−2.7 V for five pulses, −3.3 V for five pulses, and −4.5 V for five pulses) under pressure of 17.20 kPa.

FIG. 4D. Full-color electrochemiluminescent tactile visual synaptic element (ECL-TVS). Photographs of integrated R-, G-, and B-ECL-TVS at the 1^{st}, 5^{th}, 6^{th}, 10^{th}, 11^{th}, and 15^{th}V_{gs} pulses. Scale bar: 5 mm.

FIG. 5A. A Personalized finger motion monitoring and rehabilitation with a wearable electrochemiluminescent tactile visual synaptic element (ECL-TVS). Schematic illustration of finger rehabilitation exercise for index, middle, and ring fingers. The left inset photograph shows a personalized wearable finger rehabilitation ECL-TVS panel, and the right inset photograph shows a finger cap made of a lightweight rubber film with an elastomeric hemispherical top gate. Scale bar: 5 mm

FIG. 5B. A Personalized finger motion monitoring and rehabilitation with a wearable electrochemiluminescent tactile visual synaptic element (ECL-TVS). Personalized rehabilitation equipment and hand recognition algorithm process through image analysis. Scale bar: 5 cm (upper inset), 2.5 cm (lower inset).

FIG. 5C. A Personalized finger motion monitoring and rehabilitation with a wearable electrochemiluminescent tactile visual synaptic element (ECL-TVS). Schematic of the recovery processes with increments in the ‘Range of Motion’ (ROM) and contact area as rehabilitation progresses.

FIG. 5D. A Personalized finger motion monitoring and rehabilitation with a wearable electrochemiluminescent tactile visual synaptic element (ECL-TVS). Plots of the electrical output of the bioinspired full-color electrochemiluminescent tactile visual synaptic element (ECL-TVS) during the exercise for the index finger (red), middle finger (green), and ring finger (blue). The duration of tapping (t_{d, tapping})=1.5 s, interval of tapping (Δt_{tapping})=4 s, and the gate voltage at each tapping action (V_{gs, action})=−4.5 V were used during cycles of tapping action. In contrast, the erasing gate voltage (V_{gs, erase})=+3.5 V was used to return the bioinspired full-color electrochemiluminescent tactile visual synaptic element (ECL-TVS) to the initial state.

FIG. 5E. A Personalized finger motion monitoring and rehabilitation with a wearable electrochemiluminescent tactile visual synaptic element (ECL-TVS). Plots of the ECL output of luminescence during exercises corresponding to FIG. 5D. The inset photograph shows emissions of the initial and final states of the index finger (red), middle finger (green), and ring finger (blue), respectively. Scale bar: 2 mm.

FIG. 6A. Arrhythmia monitoring task based on SNN with electrochemiluminescent tactile visual synaptic element (ECL-TVS). (Left diagram) A schematic illustration of the representative five classes of ECG signals from the heart: ‘N’, ‘S’, ‘V’, ‘F’, and ‘Q’. (Middle diagram) A diagnostic flow and its visualization process based on an SNN comprising pre-(black circles) and post-neurons (colored circles) connected by synapses (yellow lines), when receiving “V” class signal. The right inset (blue dotted lines) illustrates the operation schemes that show the utilization of the bioinspired full-color electrochemiluminescent tactile visual synaptic element (ECL-TVS) within the SNN. ECL-TVS may scale V_{Pre} from various pre-neurons with the synaptic and tactile functions (w·V_{Pre}), accumulating over time as V(t) in the corresponding post-neuron. Once V(t) reaches a threshold (orange dotted line), Vₚₒₛₜ is fired and transmitted as input for ECL visualization of the diagnostic result, enabling real-time ECG monitoring. (Right diagram) Plots of STDP learning behaviors of the bioinspired full-color electrochemiluminescent tactile visual synaptic element (ECL-TVS) as a function of external pressure inputs. Δw increases when the designed voltage pulse scheme consisting of 1.5 V, 2 V, and −1.5 V with a width of 40 ms is applied to both the pre- and post-neuron with a smaller Δt and a stronger pressure. All the plots follow exponential STDP curves (red dotted lines).

FIG. 6B. Arrhythmia monitoring task based on SNN with electrochemiluminescent tactile visual synaptic element (ECL-TVS). Plots of V_{Post} over timesteps of the SNN based on ECL-TVS before and after STDP learning in response to different ECG classes at 12.51 kPa. The top panel shows the sequence of applied input classes.

FIG. 6C. Arrhythmia monitoring task based on SNN with electrochemiluminescent tactile visual synaptic element (ECL-TVS). Diagnostic accuracies of the ECG signals after the STDP learning as a function of the applied pressure. For 12.51 kPa, the estimated diagnostic accuracy is observed to achieve its optimal value (about 84%).

FIG. 6D. Arrhythmia monitoring task based on SNN with electrochemiluminescent tactile visual synaptic element (ECL-TVS). A photograph of the experimental setup for the direct visualization process of diagnostic results. Scale bar: 1 cm.

FIG. 6E. Arrhythmia monitoring task based on SNN with electrochemiluminescent tactile visual synaptic element (ECL-TVS). Photographs of ECL emission over timesteps at each post-neuron after STDP learning. Scale bar: 1 cm.

FIG. 6F. Arrhythmia monitoring task based on SNN with electrochemiluminescent tactile visual synaptic element (ECL-TVS). Plots of the ECL output over timesteps at each post-neuron, which reflects and quantifies the ECL emission in FIG. 6E.

FIG. 7. Spike encoding scheme based on Gaussian pre-neurons. (Upper diagram) A plot showing intersections (red dots) between a specific electrocardiogram (ECG) input (red dotted line) and 10 populations of pre-neurons (lines of various colors). (Lower diagram) Spike Generation (Vpre) based on population encoding scheme and a partial plot of tpre.

FIG. 8. Secondary steps for STDP learning. The upper left drawing and the upper right drawing refer to the synaptic weight compensation process before and after the post-neuron allocated as “V” receives the corresponding electrocardiogram class (in this case, “V” class). The lower left drawing and the lower left drawing refer to a synaptic weight penalty process before and after the post-neuron allocated as “V” receives a non-corresponding electrocardiogram class (in this case, “N” class).

FIG. 9. Proposal of circuit-level design of SNN based on ECL-TVS. (top view) Schematic example of SNN corresponding to the bottom view of FIG. 9. (bottom view) 1×1 synaptic composition between a pre-neuron and a post-neuron and an enlarged circuit diagram thereof.

### DETAILED DESCRIPTIONS

Advantages and features of the present disclosure, and a method of achieving the advantages and features will become apparent with reference to embodiments described later in detail together with the accompanying drawings. However, the present disclosure is not limited to the embodiments as disclosed under, but may be implemented in various different forms. Thus, these embodiments are set forth only to make the present disclosure complete, and to completely inform the scope of the present disclosure to those of ordinary skill in the technical field to which the present disclosure belongs, and the present disclosure is only defined by the scope of the claims.

For simplicity and clarity of illustration, elements in the drawings are not necessarily drawn to scale. The same reference numbers in different drawings represent the same or similar elements, and as such perform similar functionality. Further, descriptions and details of well-known steps and elements are omitted for simplicity of the description. Furthermore, in the following detailed description of the present disclosure, numerous specific details are set forth in order to provide a thorough understanding of the present disclosure. However, it will be understood that the present disclosure may be practiced without these specific details. In other instances, well-known methods, procedures, components, and circuits have not been described in detail so as not to unnecessarily obscure aspects of the present disclosure. Examples of various embodiments are illustrated and described further below. It will be understood that the description herein is not intended to limit the claims to the specific embodiments described. On the contrary, it is intended to cover alternatives, modifications, and equivalents as may be included within the spirit and scope of the present disclosure as defined by the appended claims.

A shape, a size, a ratio, an angle, a number, etc. disclosed in the drawings for illustrating embodiments of the present disclosure are illustrative, and the present disclosure is not limited thereto.

The terminology used herein is directed to the purpose of describing particular embodiments only and is not intended to be limiting of the present disclosure. As used herein, the singular constitutes “a” and “an” are intended to include the plural constitutes as well, unless the context clearly indicates otherwise. It will be further understood that the terms “comprise”, “comprising”, “include”, and “including” when used in the present disclosure, specify the presence of the stated features, integers, operations, elements, and/or components, but do not preclude the presence or addition of one or more other features, integers, operations, elements, components, and/or portions thereof. As used herein, the term “and/or” includes any and all combinations of one or more of associated listed items. Expression such as “at least one of” when preceding a list of elements may modify the entire list of elements and may not modify the individual elements of the list. In interpretation of numerical values, an error or tolerance therein may occur even when there is no explicit description thereof.

When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

When an embodiment may be implemented differently, functions or operations specified within a specific block may be performed in a different order from an order specified in a flowchart. For example, two consecutive blocks may actually be performed substantially simultaneously, or the blocks may be performed in a reverse order depending on related functions or operations.

The features of the various embodiments of the present disclosure may be partially or entirely combined with each other, and may be technically associated with each other or operate with each other. The embodiments may be implemented independently of each other and may be implemented together in an association relationship.

In interpreting a numerical value, the value is interpreted as including an error range unless there is no separate explicit description thereof. In the context of the present disclosure, the term “about” may mean about +1%, about ±2%, about ±3%, about ±4%, about ±5%, about ±6%, about ±7%, about ±8%, about ±9%, or about ±10% of a value stated herein.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “embodiments,” “examples,” “aspects, and the like should not be construed such that any aspect or design as described is superior to or advantageous over other aspects or designs.

The terms used in the description as set forth below have been selected as being general and universal in the related technical field. However, there may be other terms than the terms depending on the development and/or change of technology, convention, preference of technicians, etc. Therefore, the terms used in the description as set forth below should not be understood as limiting technical ideas, but should be understood as examples of the terms for illustrating embodiments.

In addition, it will also be understood that when a first element or layer is referred to as being present “on” a second element or layer, the first element may be disposed directly on the second element or may be disposed indirectly on the second element with a third element or layer being disposed between the first and second elements or layers. It will be understood that when a first element or layer is referred to as being “connected to”, or “coupled to” a second element or layer, the first element may be directly connected to or coupled to the second element or layer, or one or more intervening elements or layers may be present therebetween. In addition, it will also be understood that when an element or layer is referred to as being “between” two elements or layers, it may be the only element or layer between the two elements or layers, or one or more intervening elements or layers may also be present therebetween.

Further, as used herein, when a layer, film, area, plate, or the like is disposed “on” or “on a top” of another layer, film, area, plate, or the like, the former may directly contact the latter or still another layer, film, area, plate, or the like may be disposed between the former and the latter. As used herein, when a layer, film, area, plate, or the like is directly disposed “on” or “on a top” of another layer, film, area, plate, or the like, the former directly contacts the latter and still another layer, film, area, plate, or the like is not disposed between the former and the latter. Further, as used herein, when a layer, film, area, plate, or the like is disposed “below” or “under” another layer, film, area, plate, or the like, the former may directly contact the latter or still another layer, film, area, plate, or the like may be disposed between the former and the latter. As used herein, when a layer, film, area, plate, or the like is directly disposed “below” or “under” another layer, film, area, plate, or the like, the former directly contacts the latter and still another layer, film, area, plate, or the like is not disposed between the former and the latter.

In descriptions of temporal relationships, for example, temporal precedent relationships between two events such as “after”, “subsequent to”, “before”, etc., another event may occur therebetween unless “directly after”, “directly subsequent” or “directly before” is not indicated. When a certain embodiment may be implemented differently, a function or an operation specified in a specific block may occur in a different order from an order specified in a flowchart. For example, two blocks in succession may be actually performed substantially concurrently, or the two blocks may be performed in a reverse order depending on a function or operation involved.

A device for classifying and visualizing an electrocardiogram pattern according to an embodiment of the present disclosure may include: a sensor for measuring an electrocardiogram signal of a living body; an artificial neural network for receiving the electrocardiogram signal from the sensor as an input and transmitting an output via signal propagation; and a synaptic element for receiving a signal processed by the artificial neural network therefrom and visualizing an electrocardiogram pattern based on the received signal.

In the context of the present disclosure, the meaning of the term “electrocardiogram” refers to a signal generated by the electrical activity of the heart. This ECG signal includes information such as the rhythm, frequency, and path of electrical stimulation of the heartbeat. The heart condition may be analyzed based on the electrocardiogram (ECG). The electrocardiogram signal may play an important role in detecting physiological abnormalities or in diagnosing heart disease. The ECG signal is mainly composed of a P wave, a QRS complex wave, and a T wave, and each of the P wave, the QRS complex wave, and the T wave corresponds to specific cardiac activity. The electrocardiogram pattern classification and visualization device according to the present disclosure may help to more effectively evaluate a heart condition via a function of analyzing a pattern of the ECG signal and visualizing the pattern in real time.

In the context of the present disclosure, the term “electrocardiogram pattern” means a characteristic shape or change of the electrocardiogram signal. This pattern may be classified into “N”, “S”, “V”, “F”, “Q” patterns indicating normal cardiac activity. The electrocardiogram pattern includes characteristics such as periodicity, amplitude, and inter-signal interval of the signal, which may be used to evaluate whether heart activity is normal. The electrocardiogram pattern classification and visualization device according to the present disclosure may help to accurately analyze such a pattern to detect an abnormal signal associated with a specific disease.

In the context of the present disclosure, the term “electrocardiogram pattern” classification refers to a process of analyzing the electrocardiogram signal to distinguish a pattern related to a specific state or disease. This process identifies abnormal patterns such as normal heart rate patterns and arrhythmia based on the temporal and frequency characteristics of the signal. The artificial neural network of the present disclosure serves to learn and classify these patterns, and thus presents the possibility of evaluating the heart condition in real time and providing an early warning.

In the context of the present disclosure, the term “electrocardiogram visualization” means a process of displaying an electrocardiogram signal analysis result in an intuitive visual form to a user. In the present disclosure, visualization is implemented based on a color change of the electrochemiluminescent element, and each electrocardiogram pattern is expressed in a specific color. This visualization may help the user understand the heart condition at a glance and may allow the medical staff to make quick diagnostic decisions. In addition, such visual feedback has the potential to improve personal health care when being integrated into a wearable device.

The role of the sensor is to accurately measure the electrocardiogram signal of a living body and convert the measured signal into a digital signal. The sensor detects the electrical activity of the heart through an electrode attached to the skin, removes noise from the measured data and may transmit the noise-free data to the neural network. The sensor is an important component for determining the quality and accuracy of the electrocardiogram signal. In accordance with the present disclosure, the sensor may provide a basis for real-time monitoring and analysis.

In the context of the present disclosure, the term “artificial neural network” refers to an algorithm or system that imitates the structure and operation principle of a biological neural network to perform data analysis and learning. The spiking neural network (SNN) used in the present disclosure may efficiently process and learn electrocardiogram data in which a time order is important.

The role of the artificial neural network is to analyze and classify the electrocardiogram signal transmitted from the sensor. This neural network learns input data according to temporal characteristics, distinguishes normal and abnormal patterns from each other, and transmits the results to the synaptic element. In particular, the spiking neural network used in the present disclosure plays an important role in learning time-dependent characteristics of the electrocardiogram signal and visualizing the result according to the pattern.

In the context of the present disclosure, the meaning of the term input or output indicates a data flow in an electrocardiogram signal processing process. The input includes a process in which the electrocardiogram signal measured by the sensor is transmitted to the neural network, and the output includes a process in which a result processed by the neural network is input to the synaptic element which visually displays the result.

In the context of the present disclosure, the term “synaptic element” means an electrochemical element that receives a signal processed by the artificial neural network therefrom and visually outputs the signal. The synaptic element of the present disclosure is composed of an ion-gel, an organic semiconductor layer, a gate electrode, etc., and controls light emission characteristics based on an input voltage. This synaptic element is a key component that may intuitively output the analysis result, and may visualize various patterns.

The role of the synaptic element is to visually provide the analysis result to the user via electrochemical light-emission (electrochemiluminescence) based on the electrocardiogram pattern data transmitted from the neural network. This synaptic element emits light of colors such as red, green, and blue according to the size and shape of the input signal, and may provide clear feedback of distinguishing patterns from each other.

The synaptic element may receive the signal processed by the artificial neural network therefrom via the gate electrode. The signal input to the gate electrode induces an electrical change in the ion-gel and the organic semiconductor layer to adjust the magnitude and characteristics of the current flow between the source electrode and the drain electrode. Thus, the synaptic element may generate an appropriate electrochemical reaction according to the input signal and implement light emission corresponding to the electrocardiogram pattern.

In an embodiment, the synaptic element may include: a source electrode; a drain electrode horizontally spaced apart from the source electrode; an organic semiconductor layer deposited on both the source electrode and the drain electrode so as to contact both the source electrode and the drain electrode; an ion-gel layer formed on the organic semiconductor layer; and a gate electrode formed on the ion-gel layer and having a shape in which at least a portion of a lower surface thereof is convex downwardly.

In the context of the present disclosure, the dictionary meaning of “source electrode”, “drain electrode”, and “gate electrode” means a key electrical element for controlling the operation of an electronic device. The source electrode SE is a point from which the flow of electrons starts, and the drain electrode DE is a terminal point toward which electrons flow. The gate electrode GE serves to control the flow, and may control the flow of current according to an external stimulus or signal. The interaction of these three electrodes may be an important factor in determining the performance and reactivity of the synaptic element.

The functions of the “source electrode”, the “drain electrode”, and the “gate electrode” respectively perform a function of starting and ending a current flow and controlling the current flow. Specifically, the source electrode serves to supply current, and the drain electrode becomes a terminal point toward which current flows. The gate electrode regulates the current flow between the source and the drain in response to external stimuli, thereby helping the synaptic element to operate in an adapted manner to various external environments.

In particular, the role of the gate electrode is to precisely control the current flow between the source electrode and the drain electrode. The gate electrode allows an electric field to be generated in the ion-gel and the organic semiconductor layer under a voltage or stimulus input from the outside to adjust the magnitude and direction of the current. This role becomes a key element that helps the synaptic element perform a desired function in response to an external signal. In addition, the gate electrode may play an important role in adjusting the reactivity of the synaptic element based on the input signal and activating additional output characteristics such as electrochemical light-emission (electrochemiluminescence). Accordingly, the present disclosure provides the possibility of implementing a function of precisely analyzing and visualizing the electrocardiogram signal.

As long as the above-described function is performed, the shape of the gate electrode is not particularly limited. In one embodiment, the gate electrode may be formed in a shape of a partial hemisphere convex downwardly. The partial hemisphere-shaped gate electrode may have the advantage of being able to respond more uniformly to physical stimulation, especially pressure. Due to this shape, the contact area between the gate electrode and the pressure application source gradually increases when the pressure is applied thereto, such that the precise control of the electrical signal is achieved. In addition, the hemispherical shape may induce a consistent response to pressure applied at various angles, thereby providing a structure suitable for curved surfaces such as fingers or joints. Thus, the present device including the synaptic element may more accurately detect various movements of the user in association with the rehabilitation and help to generate a signal corresponding thereto.

In an embodiment, the artificial neural network may include a spiking neural network (SNN). The spiking neural network (SNN) is a neural network designed to mimic a biological neural network, and has a structure in which the transmission of signals between neurons is made in the form of temporally continuous spikes. SNN is an event-driven neural network in which the activation of neurons occurs only when a stimulus applied thereto exceeds a specific threshold, and may efficiently process the temporal pattern and order of the input signals. These characteristics are suitable for analyzing continuous data that changes over time, such as electrocardiogram, and may provide high energy efficiency even in environments where computational resources are limited. Therefore, the SNN is likely to act as a key component configured to learn and classify the patterns of the electrocardiogram signal in accordance with the present disclosure.

The ion-gel layer may include a polymer matrix; an ionic liquid dispersed in the polymer matrix and having electrical conductivity; one or more luminophores dispersed in the polymer matrix and generating electrochemical luminescence in response to the electrical stimulation; and a co-reactant transferring electrons to the luminophores in response to the electrical stimulation.

In the context of the present disclosure, the term “organic semiconductor” refers to an organic compound-based semiconductor material, has intermediate properties of conductivity and insulation, and includes properties of responding to electrical, optical, and chemical stimuli. Since the organic semiconductor is generally light and flexible and is able to be processed into a semiconductor element at low temperatures, the organic semiconductor is highly likely to be used in applications such as wearable electronic devices and bio-signal sensing devices. This characteristic may contribute to allowing the synaptic element of the present disclosure to precisely analyze and output the electrocardiogram signal.

The role of the “organic semiconductor layer” is to provide a current path between the source electrode and the drain electrode, and to change electrical characteristics according to an electrical signal input from the gate electrode. The organic semiconductor layer sensitively detects an electric field induced by the gate electrode through interaction with the ion-gel, and adjusts the flow of current based on the detection result. In this process, the organic semiconductor layer may implement various synaptic characteristics such as amplification, storage, and transmission of an input signal, and may also affect the intensity and uniformity of light emission generated from the electrochemical reaction. This role may help to increase the visualization and analysis accuracy of the electrocardiogram signal.

As long as the above-described function is performed, the material of the organic semiconductor layer is not particularly limited. In an embodiment, the organic semiconductor layer may include poly(3-hexylthiophene) (P3HT). P3HT (poly(3-hexylthiophene)) is known as an organic semiconductor material having excellent conductivity, easily subjected to processing, and flexible characteristics. These characteristics are particularly suitable for the flexible electronic devices or wearable devices, and may contribute to improving the durability of the synaptic element. P3HT also has high charge mobility thereby enabling a fast electrical response to external stimuli.

In the context of the present disclosure, the term “ion-gel” means a gel-state material formed by dispersing the ionic liquid in the polymer matrix. The ion-gel has both ionic conductivity and flexibility, and is characterized in that it may express electrochemical properties in response to electrical stimulation. In particular, the ion-gel provides high chemical stability and a wide electrochemical window, so that it may operate stably in an electrochemical device. In the present disclosure, the ion-gel may play a key role in interacting with the luminescent body in response to electrical stimulation.

The role of the ion-gel layer is to induce the movement of ions and an electrochemical reaction based on an electrical signal input from the gate electrode to generate electrochemical light-emission (electrochemiluminescence). The ion-gel layer sensitively senses an electrical signal and activates the luminophores and the co-reactant contained therein to help emit light of a specific wavelength. In addition, the ion-gel layer may adjust electrical characteristics at an interface with the organic semiconductor layer, and may contribute to adjusting light emission intensity and color according to an input signal. This role performs an important function of visually outputting the pattern of the electrocardiogram signal to help users and medical staff to easily interpret the pattern of the electrocardiogram signal.

As long as the above-described function is performed, the configuration included in the ion-gel layer is not particularly limited. In an embodiment, the ion-gel layer may include a polymer matrix; an ionic liquid dispersed in the polymer matrix and having electrical conductivity; one or more luminophores dispersed in the polymer matrix and generating electrochemical luminescence in response to the electrical stimulation; and a co-reactant transferring electrons to the luminophores in response to the electrical stimulation.

In the context of the present disclosure, the dictionary meaning of “polymer matrix” refers to a continuous network structure made of a polymer material. This structure has the function of containing or dispersing various components therein and may provide mechanical and chemical stability. The polymer matrix may contribute to preserving the functionality of the synaptic element by effectively containing the components such as the ionic liquid and luminophores therein. The flexibility and durability of the synaptic element may be improved based on various polymer combinations.

The role of the “polymer matrix” is to provide an environment in which the ionic liquid and the luminophores are stably dispersed and the ionic liquid and the luminophores may respond to electrochemical stimulation. The polymer matrix may act as a medium that allows the ion movement while supporting the physical structure of the synaptic element. In addition, the polymer matrix may contribute to maintaining electrical performance while absorbing structural deformation according to the external pressure or stimulus, thereby increasing the continuous performance and durability of the synaptic element.

As long as the above-described function is performed, the material of the polymer matrix is not particularly limited. In an embodiment, the polymer matrix may include P(VDF-HFP) (poly(vinylidene fluoride-hexafluoropropylene)). P(VDF-HFP) (poly(vinylidene fluoride-hexafluoropropylene)) is a polymer with high flexibility and excellent electrical and chemical stability, and may be suitable for applications in electronic devices. This material effectively disperses the ionic liquid and the luminophores therein and may help maintain reactivity to electrochemical stimuli. In addition, the P(VDF-HFP) provides flexible properties while maintaining mechanical strength, thereby increasing resistance to pressure or bending. Thus, the synaptic element may operate stably even under repeated stimulation, and performance degradation of the synaptic element may be prevented even in long-term use.

In the context of the present disclosure, the dictionary definition of “ionic liquid” is an ionic material that exists in a liquid state at room temperature, and has low volatility and stable properties while having electrical conductivity unlike a general solvent. Due to these characteristics, in electronic devices, the ionic liquid may be advantageously utilized to promote electrochemical reactions or control electrical properties. There is a possibility of optimizing the performance of the synaptic element via a combination of various ionic liquids.

The role of the “ionic liquid” is to promote ion migration in the polymer matrix and induce an electrochemical reaction according to electrical stimulation. The ionic liquid is a medium that transmits electrical stimulation, and may interact with luminophore to induce a luminescent reaction. This may increase the sensitivity of the synaptic element and contribute to activating the process in which the electrical signal is converted into visual feedback. In addition, the ionic liquid may help to improve efficiency and stability of the synaptic element.

As long as the above-described function is performed, the material of the ionic liquid is not particularly limited. In an embodiment, the ionic liquid may include 1-butyl-1-methylpyrrolidinium bis(trifluoromethylsulfonyl)imide ([PYR₁₄][TFSI]). 1-butyl-1-methylpyrrolidinium bis(trifluoromethylsulfonyl)imide ([PYR₁₄][TFSI]) is an ionic liquid having excellent electrochemical stability and a wide voltage window, and may be suitable for electronic devices. [PYR₁₄][TFSI] promotes ion migration, thereby enabling sensitive reactions to electrical stimuli, and may induce efficient luminescence via the interaction with the luminophores.

In the context of the present disclosure, the dictionary meaning of “electrochemical luminescence” refers to a phenomenon in which light emits via the electrochemical reaction induced under the electrical stimulus. In this process, electrochemically activated luminophore molecules undergo oxidation or reduction reactions, and then transition to an excited state, and then return to a ground state to emit photons. In the electrochemical light-emission (electrochemiluminescence), the ionic material and a reactant in the polymer matrix organically react with each other to generate light of a specific wavelength, thereby providing high selectivity and a signal-to-noise ratio.

In the context of the present disclosure, the dictionary meaning of “luminophore” is a material that emits light under electron stimulation thereto, and is mainly a component responsible for a light emitting function in an electronic device. The luminophores emit energy by interacting with electrons under certain conditions, and in this process, the luminophores may generate light of various colors. The luminophores may be used as an important factor related to the luminous efficiency of the synaptic element.

The role of the “luminophore” is to induce light emission in response to electrical stimulation. The luminophores interact with the ionic liquid and the co-reactant and emit light via collisions with electrons. In this process, the user may visually check the tactile stimulus. Thus, an environment in which the user may receive feedback on his or her movement in the rehabilitation process in real time may be provided. In addition, the luminophores may determine the color of the emitted light to produce various visual effects.

As long as the above-described function is performed, the material of the luminophore is not particularly limited. In an embodiment, the luminophore may include one or more materials selected from the group consisting of Ru(bpy)₃Cl₂, Ir(dfppy)₂(bpy)PF₆, and Ir(diFppy)₂(pic) (Firpic). Ru(bpy)₃Cl₂, Ir(dfppy)₂(bpy)PF₆, and Ir(diFppy)₂(pic) (Firpic) are representative luminophores that induce red, green, and blue light emission, respectively, and may realize various light emission colors. These luminophores react sensitively to electrical stimuli and emit light through collisions with electrons, and through this process, visual feedback may be provided to the user. In particular, since each luminophore has its own oxidation potential and luminescence characteristics, the driving voltage and emission spectrum of the synaptic element may be controlled to realize various visual effects.

In the context of the present disclosure, the dictionary meaning of “co-reactant” refers to a material that promotes or assists reactivity in a chemical reaction process. In the electrochemiluminescent system, the co-reactant together with the luminophore serves to activate the light emission reaction, and may be utilized as one of the essential components for efficient light emission.

The role of the co-reactant is to transfer electrons to luminophores to promote a luminescence reaction. When an electrical stimulus is applied to the co-reactant, the co-reactant may interact with the luminophore to accelerate the electron transfer process, thereby inducing light emission. This process may contribute to increasing the overall luminous efficiency of the synaptic element, and may serve as a basis for a user to receive visual feedback more clearly and quickly.

As long as the above-described function is performed, the material of the co-reactant is not particularly limited. In an embodiment, the co-reactant may include tripropylamine (TPrA). The tripropylamine (TPrA) is a co-reactant, and may play an important role in accelerating a luminescence reaction by transferring electrons to luminophores when an electrical stimulus is applied thereto. TPrA produces active radicals during the oxidation process, and these radicals interact with the luminophores to promote electron transfer. This process may greatly improve the efficiency of electrochemical light-emission (electrochemiluminescence), and may contribute to optimizing light emission intensity and reaction time.

In an embodiment, the electrocardiogram pattern classification and visualization device may classify the ECG patterns into “N” (Normal), “S” (Supraventricular Arrhythmia), “V” (Ventricular Arrhythmia), “F” (Fusion Beat), and “Q” (Unknown or Noisy Beats).

In the present disclosure, the “N” (Normal) pattern means a form of an electrocardiogram signal occurring in normal heart activity. This pattern is composed of a regular rhythm, a stable P wave, a QRS complex wave, and an T wave, and indicate a state in which the heartbeat is constant and there is no abnormality. The “N” pattern reflects a process in which electrical stimulation of the heart is normally generated and conducted, and may be a criterion for determining a healthy heart rate state.

In the present disclosure, the “S” (Supraventricular Arrhythmia) pattern refers to a form of an electrocardiogram signal caused by arrhythmia originating in the atrium. This pattern may be characterized as an abnormal P wave or a narrow QRS complex wave, and may be accompanied by a rapid increase in heart rate or irregularity. The “S” pattern may be interpreted as a sign of a supraventricular arrhythmia, such as atrial fibrillation or atrial flutter.

In the present disclosure, the “V” (Ventricular Arrhythmia) pattern means a form of an electrocardiogram signal caused by arrhythmia originating in the ventricle. This pattern may be characterized as an abnormally wide QRS complex wave and irregular heartbeat rhythm, and there is often no correlation between P and QRS waves. The “V” pattern may indicate a lethal condition, such as ventricular tachycardia or ventricular fibrillation. Thus, the “V” pattern indicates the immediate action to be taken.

In the present disclosure, the “F” (Fusion Beat) pattern refers to a form of an electrocardiogram signal in which a normal heart rate signal and a ventricular origin signal are fused with each other. This pattern occurs in the form of a mixture of normal QRS and abnormal QRS, and the QRS wave may have a slightly wide or deformed shape. The “F” pattern may be observed in a state such as ventricular tachycardia, and may be used to determine whether an arrhythmia is progressing.

In the present disclosure, the meaning of the “Q” (Unknown or Noise Beats) pattern means an electrocardiogram signal that is not accurately classified due to signal interference or noise. This pattern represents a case in which the shape of the signal is unclear or contains noise, making it difficult to classify the state indicated thereby as a specific state. The “Q” pattern may reflect situations in which data quality verification or further review is required.

In an embodiment, the synaptic element of the electrocardiogram pattern classification and visualization device may visualize different colors based on the different electrocardiogram patterns. Thus, the user may intuitively check the state of the electrocardiogram signal, and may easily understand the health state without complex data interpretation. This visualization may help medical staff and others make quick and accurate diagnostic decisions, enabling rapid response, especially in urgent situations. In addition, color-based feedback provides real-time information to users, thereby having the potential to contribute to continuous health care and monitoring of predictable risk factors. The synaptic element is implemented in a wearable form, thereby increasing portability and accessibility such that the electrocardiogram pattern classification and visualization device may be utilized in various environments.

An advantage of the electrocardiogram pattern classification and visualization device according to an embodiment of the present disclosure is that the electrocardiogram signal may be analyzed in real time and intuitively visualized. Thus, complex electrocardiogram data may be delivered easily and quickly, and not only medical staff but also general users may reduce the burden of data interpretation. In addition, the electrocardiogram pattern classification and visualization device may be used in various environments on the basis of low power consumption and high portability, and provides the possibility of supporting continuous and personalized health care via the integration thereof with wearable devices.

Further, the electrocardiogram diagnosis method according to an embodiment of the present disclosure may include analyzing the electrocardiogram of a living body using the electrocardiogram pattern classification and visualization device according to the present disclosure. This method may quickly detect abnormalities in electrocardiogram signals and help identify heart diseases such as arrhythmia early. This method assists the medical staffs diagnosis process and has the potential to provide an opportunity for users to monitor their health status in real time. Furthermore, this method may alleviate the time and material burden that may occur in the existing diagnosis process via a non-invasive and user-friendly approach.

Hereinafter, examples of the present disclosure will be described. However, the examples as described below are only some implementations of the present disclosure, and the scope of the present disclosure is not limited to the following examples.

### [Bioinspired Full-Color Electrochemiluminescent Tactile Visual Synaptic Element (ECL-TVS)]

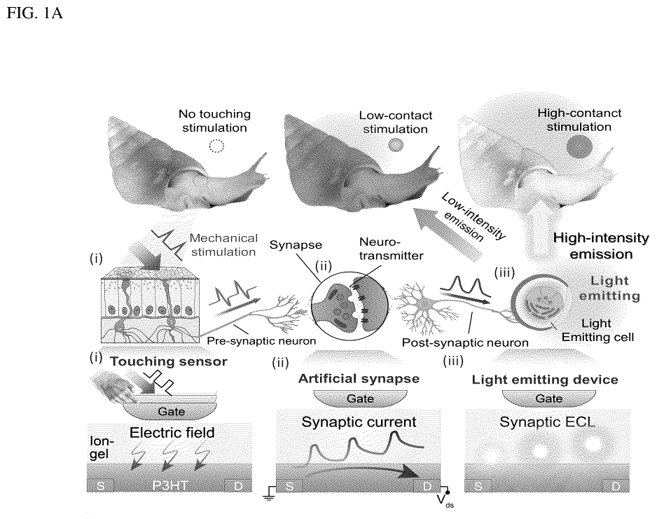

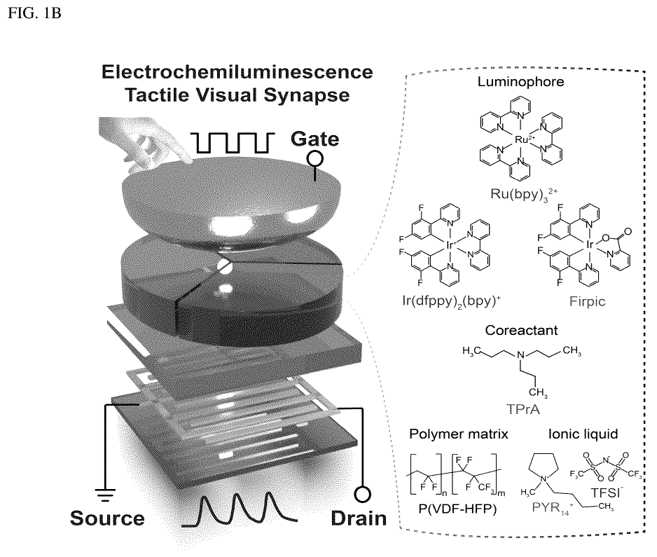

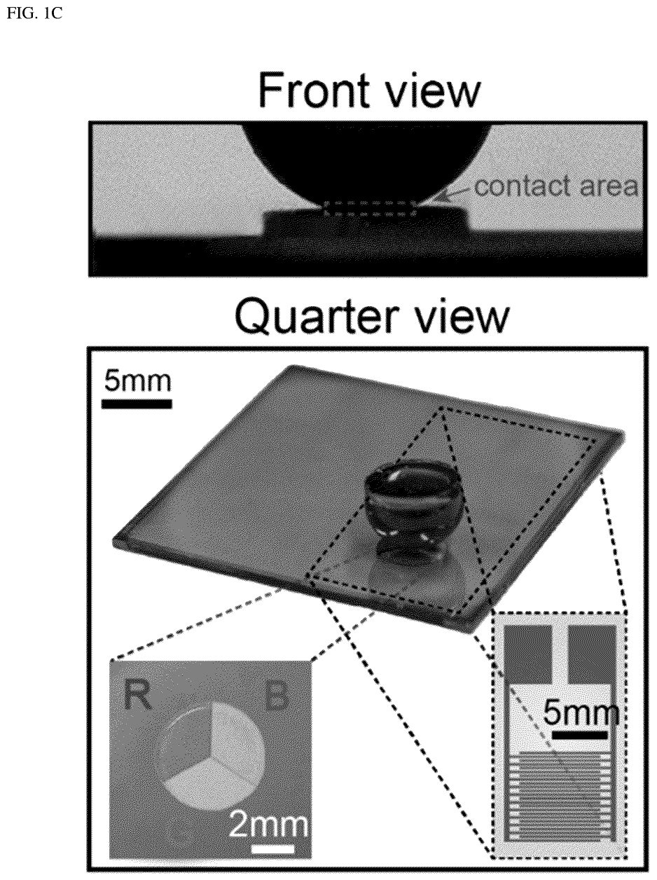

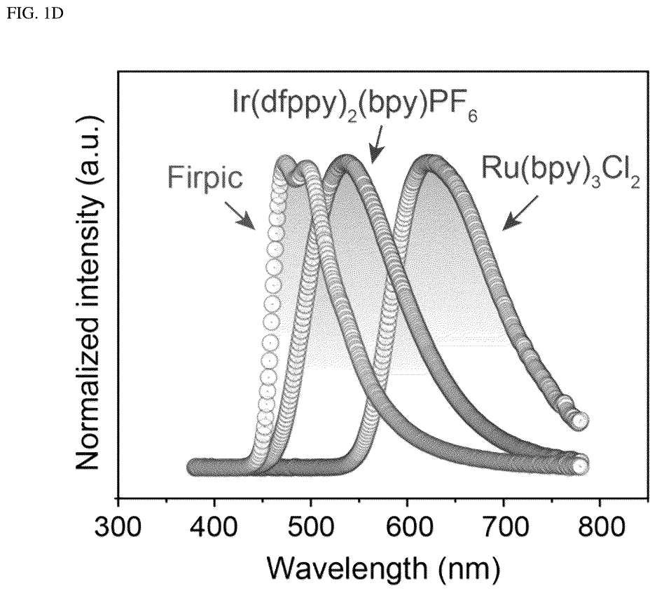

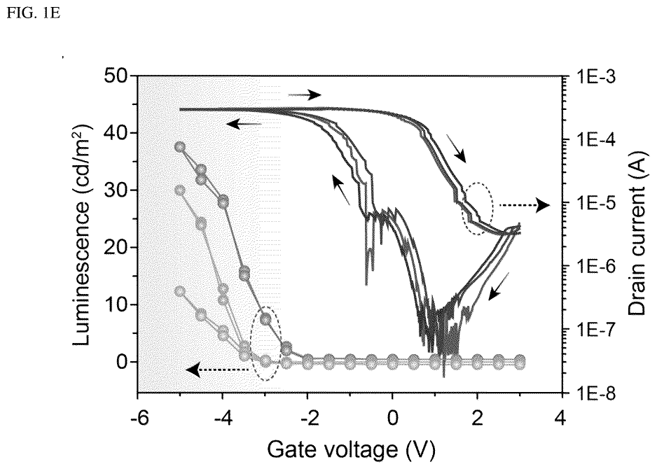

Hinea brasiliana, a glowing sea snail, processes a tactile sensory synaptic display that protects it from predators, as schematically shown in the upper diagram of FIG. 1A. In response to an external physical stimulus, the tactile sensory receptor (epidermal mechanoreceptor) evokes the action potential of the cells ((i) in the upper diagram of FIG. 1A). The receptor releases neurotransmitters and transmits signals through the nervous system connected by synapses between pre- and post-synaptic neurons ((ii) in the upper diagram of FIG. 1A)). The signals are in turn transmitted to a light-emitting cell (the photocyte) in the epithelial tissue, secreting intercellular enzymes (luciferase), inducing the characteristic chemiluminescence (CL) ((iii) in the upper diagram of FIG. 1A). When the snail encounters an attack from a predator that results in mechanical taps on the surface, it emits green CL based on the mechanism described previously. Interestingly, the light intensity is proportional to the strength of the tapping (both magnitude and frequency).

Inspired by the touch-responsive synaptic CL of the sea snail, the inventors of the present disclosure developed a full-color single-element electrochemiluminescent (ECL) tactile visual synaptic element (ECL-TVS) based on a top-gated bottom contact OECT with a mechanically deformable elastomeric top gate, as schematically shown in FIG. 1B. The ECL-TVS is composed of interdigitated indium tin oxide (ITO) source/drain electrodes with a channel length and width of 120 m and 90 mm, respectively, and a thin poly(3-hexylthiophene-2,5-diyl) (P3HT) film of approximately 50 nm in thickness. On top of the P3HT layer, an approximately 750 m-thick ion-gel, which is composed of a poly(vinylidene fluoride-co-hexafluoropropylene) [P(VDF-HFP)] matrix and an ionic liquid-1-butyl-1-methylpyrrolidinium bis(trifluoromethyl sulfonyl)imide ([PYR₁₄][TFSI]) containing co-reactant tripropylamine (TPrA) and luminophores, is placed using the ‘cut and stick’ method 34. The element utilizes three representative luminophores: Ru(bpy)₃Cl₂, Ir(dfppy)₂(bpy)PF₆, and Ir(diFppy)₂(pic) (Firpic) for red, green, and blue ECL, respectively. The top layer contains a mechanically deformable, hemisphere-shaped elastomeric polydimethylsiloxane (PDMS) gate, coated with a thin Cr/Au film (3/60 nm), positioned on the ion-gel layer.

The electrochemiluminescent tactile visual synaptic elements (ECL-TVS) emulates the tactile visual synapse of a sea snail in a single-element platform. As depicted in (i) in the lower diagram of FIG. 1A, a mechanically deformable hemisphere top gate electrode may serve as a mechanoreceptor with which the gate field is exerted on the gate. The source-to-drain current through the P3HT channel corresponds to the synaptic properties observed in sea snails, as schematically shown in (ii) in the lower diagram of FIG. 1A. Finally, the ECL in the ion-gel replicates the light emission observed in the phagocytes of sea snails, as depicted in (iii) in the lower diagram of FIG. 1A. Direct full-color ECL visualization of the synaptic activity represented by the stimuli-dependent P3HT channel current was achieved by the proper formulation of the ionic liquid ([PYR₁₄][TFSI]), co-reactant (TPrA), and luminophores (R, G, and B) in an electrically robust dielectric polymer, P(VDF-HFP) matrix. The homogeneous mixing of both reactant and luminophores in an ion-gel was identified using X-ray photoelectron spectroscopy (XPS) as well as 2D grazing incident wide-angle X-ray scattering (GIWAXS).

The contact area of the hemispherical top gate on an ion-gel was controlled by deforming the elastomeric top gate with pressure. As shown in the top photograph of FIG. 1C, the top gate was readily deformed under gentle pressure (about 12.51 kPa), resulting in a contact diameter of about 2 mm. The quarter view in the bottom photograph shown in FIG. 1C depicts the electrochemiluminescent tactile visual synaptic elements (ECL-TVS) with a hemisphere-shaped gate electrode. The left inset displays the photo of a circular-shaped ion-gel layer with a 5 mm diameter containing three pieces of ion-gels with red, green, and blue luminophores. The right inset in FIG. 1C shows an interdigitated source/drain (S/D) electrode pattern. The cross-section of the electrochemiluminescent tactile visual synaptic elements (ECL-TVS) was examined using a scanning electron microscope (SEM) combined with energy dispersive X-ray (EDX), and the results show that all the constituent layers are uniformly laminated. Additionally, EDX results obtained from the top view of the ion-gel show that the corresponding luminophores are evenly dispersed in the gel.

In the proposed electrochemiluminescent tactile visual synaptic element (ECL-TVS), the electrical synaptic activity arising from an electrical input of a top gate is represented by the lateral P3HT channel current, while the visualization of the synaptic activity that results from the electrochemical reaction in the ion-gel sandwiched between the top gate and P3HT channel. The three electrochemiluminescent tactile visual synaptic elements (ECL-TVS) with R-, G-, and B-luminophores successfully emit the characteristic red, green, and blue ECL, respectively, when a pressure of approximately 12.51 kPa was exerted on the hemispherical top gate. The ECL spectra illustrated in FIG. 1D confirm the full-color emission from the electrochemiluminescent tactile visual synaptic elements (ECL-TVS) with the wavelengths at the maximum intensity of approximately 620 nm, 543 nm, and 474, 493 nm for R-, G-, and B-luminophores, respectively. Moreover, the inventors of the present disclosure examined the electrical transfer and luminance characteristics of the electrochemiluminescent tactile visual synaptic elements (ECL-TVS) as a function of the gate voltage, and the results are presented in FIG. 1E. All three types of the bioinspired full-color electrochemiluminescent tactile visual synaptic elements (ECL-TVS) demonstrate similar transfer curves, with an ON/OFF current ratio of approximately 1.13×10⁴. Moreover, they show electrical hysteresis owing to the presence of the ionic liquids [PYR₁₄][TFSI] in the ion-gels. The maximum brightness of approximately 37.82 cd/m² was achieved in the electrochemiluminescent tactile visual synaptic elements (ECL-TVS) (FIG. 1E). The brightness was higher than that recently reported using solid-based ECL gel (about 25 cd/m²). Moreover, the emission spectra of R, G, and B ECL recorded during forward and reverse voltage sweep cycles confirm the reliability of ECL emission in the present proposed element.

As shown in FIG. 1F, the electrical threshold voltages for the R-, G-, and B-ECL-TVS elements are approximately −0.46 V, −0.53 V, and −0.37 V, respectively. The corresponding threshold voltages for light emission are approximately −2.47 V, −3.43 V, and −2.81 V. The results confirm that the threshold voltages for R, G, and B emissions depend on the oxidation potentials of the co-reactant and luminophores. For further verification, the oxidation potentials of the co-reactant (TPrA) and R-, G-, and B-luminophores were measured using cyclic voltammetry, and the oxidation potentials were identified as +1.98, +2.33, +2.82, and +2.52 V, respectively. The values are consistent with those observed in the electrochemiluminescent tactile visual synaptic elements (ECL-TVS) (FIG. 1F). Notably, the ionic liquid [PYR₁₄][TFSI] employed in the bioinspired full-color electrochemiluminescent tactile visual synaptic elements (ECL-TVS) exhibits a wider chemical stability window than those of other ionic liquid-based ion-gels. The inventors of the present disclosure conducted a comparative analysis with 11 additional ionic liquids commonly used for ionic element applications. The electrochemical stability windows of the ion-gels were determined using cyclic voltammetry. Among the tested ionic liquids, [PYR₁₄][TFSI] exhibited the largest electrochemical window of approximately 5 V, enabling for the stable operation of R, G, and B ECL, as explicitly described later.

### [Analysis of the Operational Mechanism in an Electrochemiluminescent Tactile Visual Synaptic Element (ECL-TVS)]

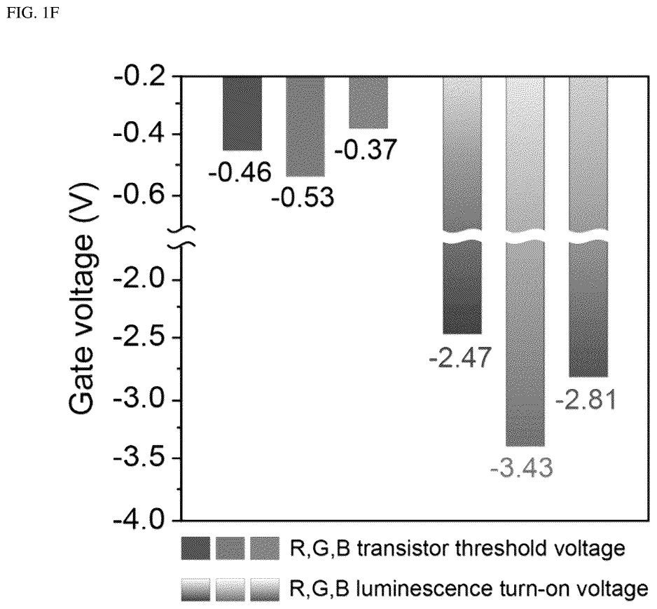

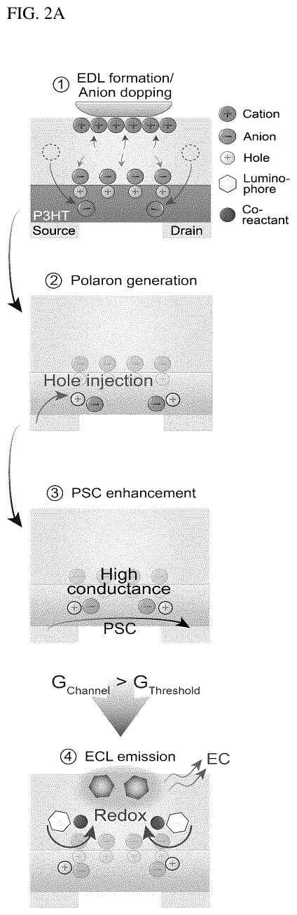

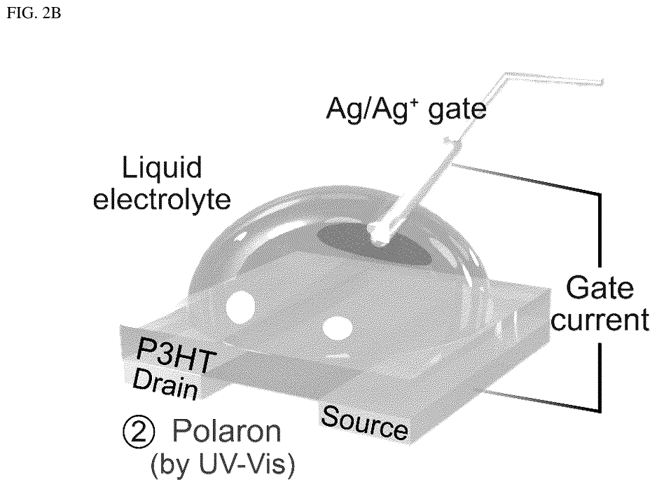

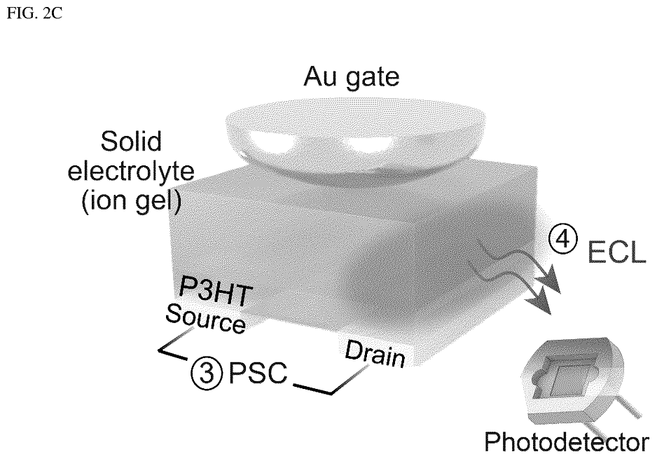

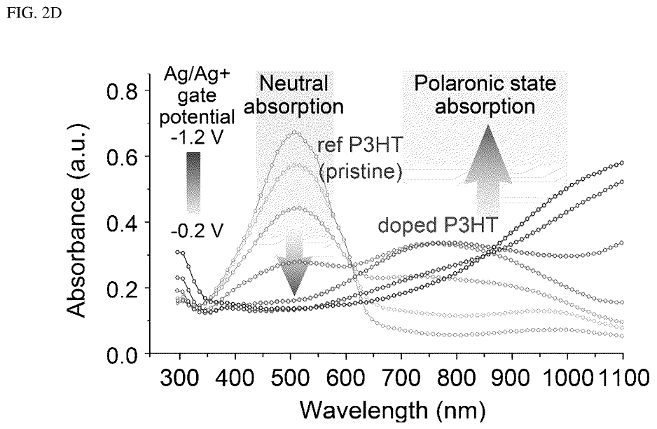

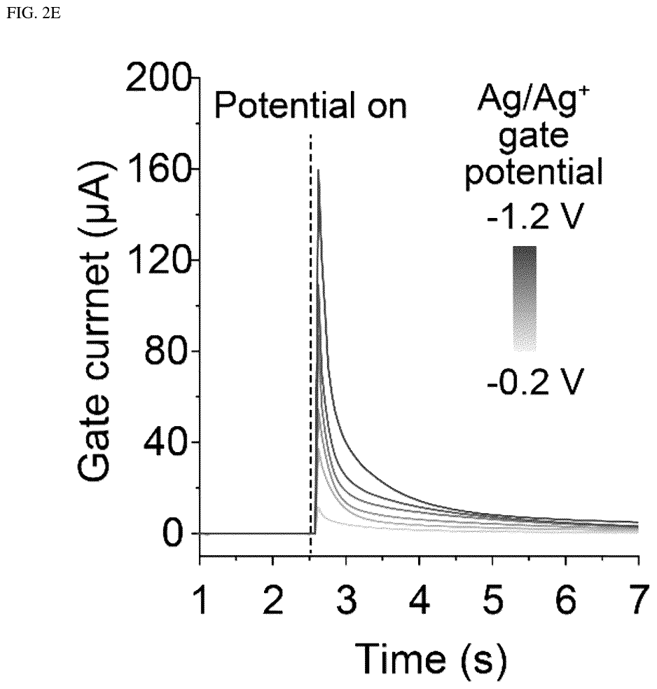

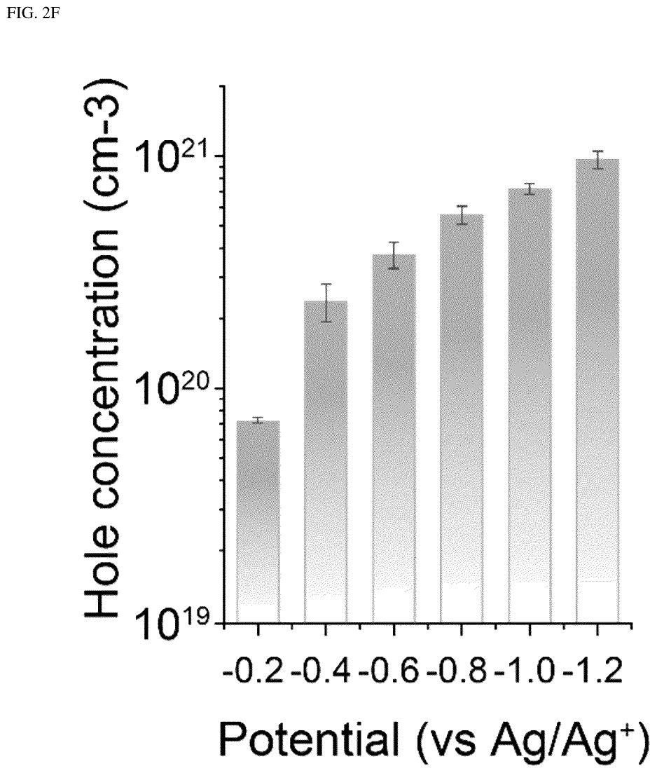

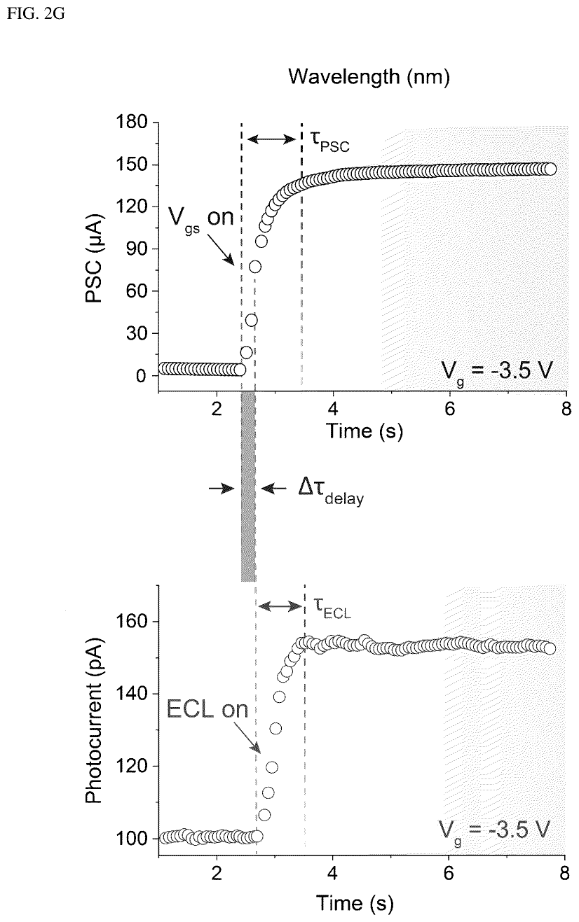

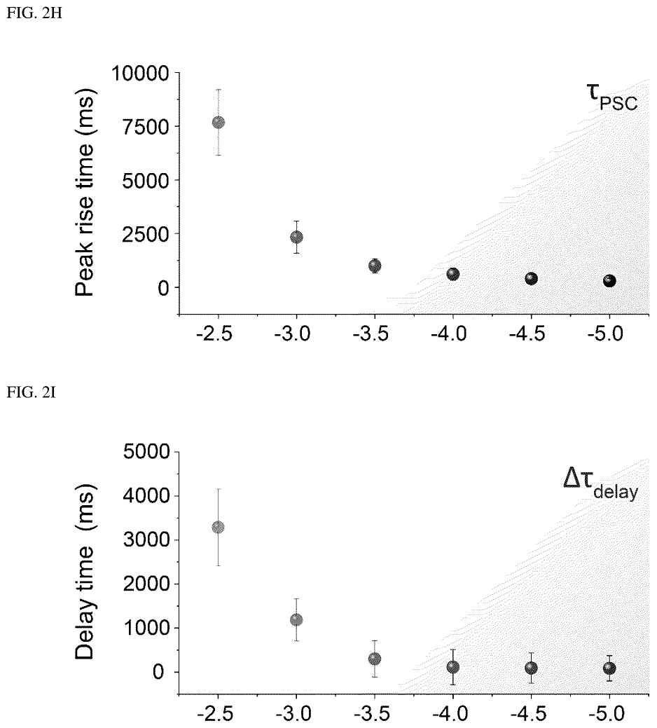

The synaptic current from the P3HT channel and ECL in the ionic gel associated with the mechanical touch events occurred as follows: 1. Electric double layer (EDL) formation and anion doping into the P3HT channel, 2. Polaron generation, 3. Enhanced conductance of the P3HT channel, and 4. ECL emission in the ion-gel correlated with the enhanced P3HT channel conductance, as schematically illustrated in FIG. 2a. When a negative pulse was imposed on an electrochemiluminescent tactile visual synaptic element (ECL-TVS), EDL was instantly developed at the ionic gel, including [PYR₁₄][TFSI] with a P3HT channel, followed by the doping of TFSI anions into the P3HT channel. Simultaneously, holes were injected from an ITO drain electrode, producing polaron pairs of holes and TFSI anions. The P3HT channel conductance was increased because of the developed polarons, enhancing the source-drain current (channel current, Ids) at a given drain-source voltage of −0.1 V. When consecutive gate pulses were imposed on the bioinspired full-color electrochemiluminescent tactile visual synaptic element (ECL-TVS), the channel current was increased in response to the number of the pulses because of the cumulative increase in the accumulated number of polarons in the P3HT channel, resulting in the characteristic synaptic LTP behavior for the electrical synaptic mechanism and basic performance of the bioinspired full-color electrochemiluminescent tactile visual synaptic element (ECL-TVS).

The underlying ECL mechanism in the bioinspired full-color electrochemiluminescent tactile visual synaptic element (ECL-TVS) is intricately related to the electrical synaptic characteristics of the bioinspired full-color electrochemiluminescent tactile visual synaptic element (ECL-TVS). Especially, ECL originates from a redox reaction of luminophores added into the ion-gel. To initiate the redox reaction of the given luminophores, a capacitive voltage greater than the threshold redox voltage of the luminophores should be applied at the gate with respect to the drain. Second, since the redox reaction occurred at the interface between the ion-gel and the P3HT channel, the P3HT channel should supply a sufficient number of hole carriers to facilitate the interface redox reaction, which should correlate with the electrical conductance of the ion-gel-gated P3HT channel. The strengthened intensity of ECL at a gate voltage greater than the threshold voltage of the luminophores implies a close link between the synaptic behavior (i.e. the electrical element characteristics) with the gate pulse-dependent ECL behavior, enabling direct visualization of the electrical synaptic properties.

To quantitatively assess the mechanistic details of the ECL synaptic behaviors, the inventors of the present disclosure first performed an in-situ spectroelectrochemical experiment to support the present speculation of the ECL dependent upon the synaptic P3HT channel current. The spectral signatures of polaronic states in the P3HT channel were monitored in real time upon gate bias ranging from −0.2 to −1.2 V vs Ag/Ag+ reference electrode in an OECT with a liquid electrolyte containing [PYR₁₄][TFSI] in acetonitrile. As shown in FIG. 2D, when the applied gate voltage was increased negatively, the broad absorption peak from 750 nm to 1100 nm, corresponding to the increase in the polaronic absorption, while the absorption peak of neutral P3HT with maximum intensity at approximately 500 nm decreased. Notably, the spectroelectrochemical analysis was conducted in an OECT without ECL reactants (luminophore and TPrA) when resolving the polaronic absorption feature particularly when a large negative gate voltage is applied to eliminate the effect of the ECL reactants on UV-vis absorption. The inventors of the present disclosure found that the absorption also occurred in the UV-vis range due to the ECL redox reaction and the absorption of Ru(bpy)₃Cl₂, particularly at WE potentials greater than 0.8 V vs. Ag/AgCl.

Furthermore, the inventors of the present disclosure measured the gate current to quantitatively calculate the hole concentration (number of holes injected per unit volume) in response to the gate potentials ranging from −0.2 to −1.2 V. The results are shown in FIG. 2E. The hole concentration was calculated by integrating the gate current over time using the following relation:

$$hole concentration = \frac{1}{e \cdot W \cdot L \cdot d} {\int }_{{t}_{1}}^{{t}_{2}} {I ( t )}_{gate} \text{?} dt \qquad [ Equation ]$$

$$\text{?} \text{indicates text missing or illegible when filed}$$

where e denotes the elementary charge and W, L, and d denote the channel width, length, and thickness, respectively. As shown in FIG. 2F, similar to the results from the spectroelectrochemical measurements (FIG. 2D), the number of generated hole polarons increased with the gate potential. The hole carrier concentration reached approximately 10²¹/cm³ at a gate bias of −1.2 V, comparable with the hole concentration typically observed in bulk-doped channels of the OECTs. Therefore, the inventors of the present disclosure may suspect that a large degree of doping of the P3HT channel is a necessary condition for the observed ECL emission to occur in the bioinspired full-color electrochemiluminescent tactile visual synaptic elements (ECL-TVS).

In-situ monitoring of the source-drain current and ECL intensity with time was performed as a function of gate bias from −2.5 to −5 V in an electrochemiluminescent tactile visual synaptic element (ECL-TVS), as schematically shown in FIG. 2C. The gate bias values were chosen to be greater than the threshold voltage of a red luminophore of −2.3 V, which is within the electrochemical stability window of the ion-gel used (about 5V). Prior to the analysis of the correlation between the source-drain current and ECL intensity in the electrochemiluminescent tactile visual synaptic element (ECL-TVS), the hole concentration of a P3HT channel of the bioinspired full-color electrochemiluminescent tactile visual synaptic element (ECL-TVS) at a gate bias of −1.2 V was calculated with the gate current at the gate bias combined using the equation above. The hole concentration at the gate bias of −1.2 V was approximately 10²¹/cm³, comparable with the value calculated from the OECT with a liquid electrolyte. The results validate that the TFSI anion doping characteristics involving the polaron generation in the bioinspired full-color electrochemiluminescent tactile visual synaptic element (ECL-TVS) with a solid-type ion-gel electrolyte are almost identical to those of the OECT with a liquid electrolyte.

Both source-drain current and ECL were increased with time at a given gate voltage of −3.5 V, as shown in FIGS. 2G and 2H, respectively. The rate at which the PSC saturated increased with the gate bias, as shown in FIG. 2I, which is qualitatively consistent with the in-situ spectroelectrochemistry results that directly indicate the degree of polaron formation by TFSI-doping. The rate at which ECL emission intensity saturated also increased with the gate bias, as shown in FIG. 2K. Overall, the results from the spectroelectrochemistry, PSC, and ECL all indicate that ECL is closely related to the P3HT channel conductance governed by the polaron density generated from TFSI-ion doping and the level of hole injection from drain electrode.

Interestingly, the inventors of the present disclosure observed that there was a delay in the onset of ECL emission (FIG. 2H), Δτ_delay, from the onset of PSC channel at a given gate bias (FIG. 2G). More specifically, ECL occurred at approximately 300.4 ms after the channel current onset at the gate bias of −3.5 V. This reflects the extra time required to reach a certain channel conductance (and therefore the polaron density in the channel) before initiating ECL emission. Δτ_delay decreased with the gate bias (FIG. 2J), until ECL emission was almost simultaneously activated from the gate bias of −4 V, enabling the direct visualization of the synaptic behavior via ECL. Additionally, the origin of the current between the top gate and the bottom source/drain electrode (Igs) of the electrochemiluminescent tactile visual synaptic element (ECL-TVS), which is responsible for ECL, was further analyzed with a set of OECTs with different components.

### [Visualization of the Tactile Synaptic Activity in an Electrochemiluminescent Tactile Visual Synaptic Element (ECL-TVS)]

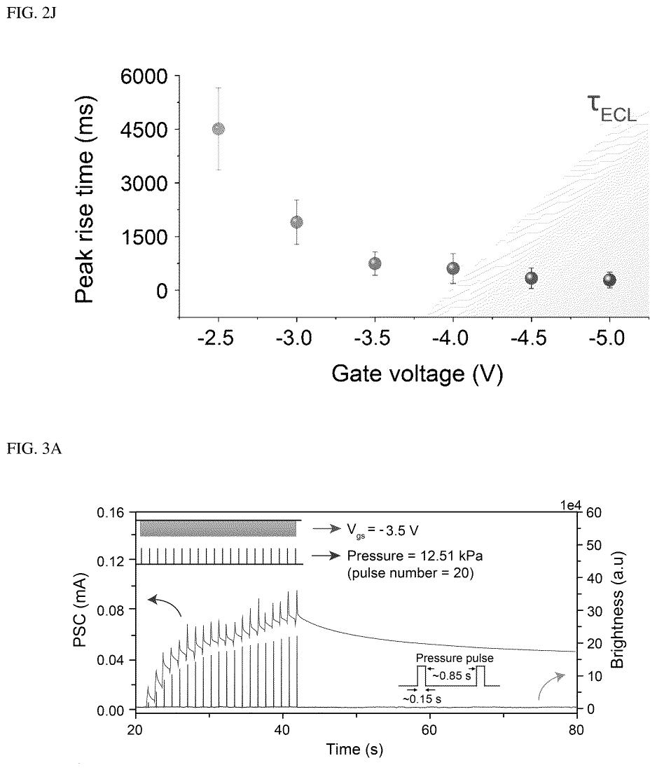

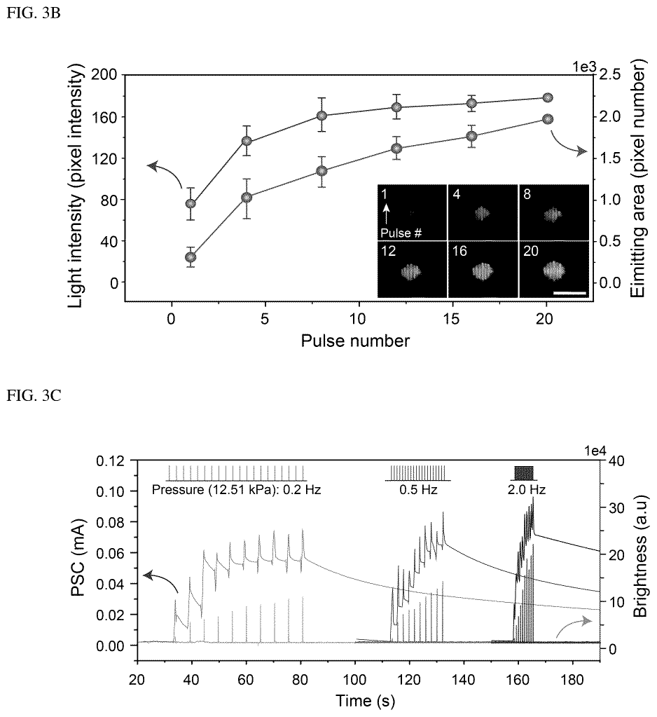

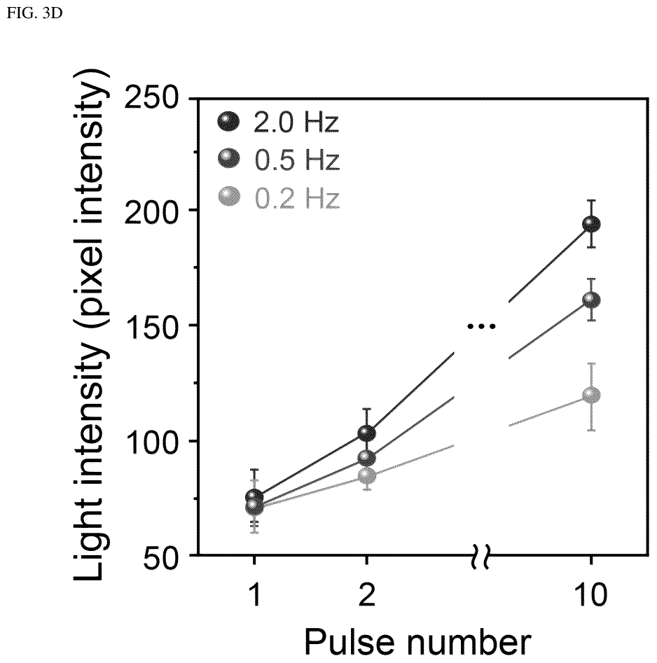

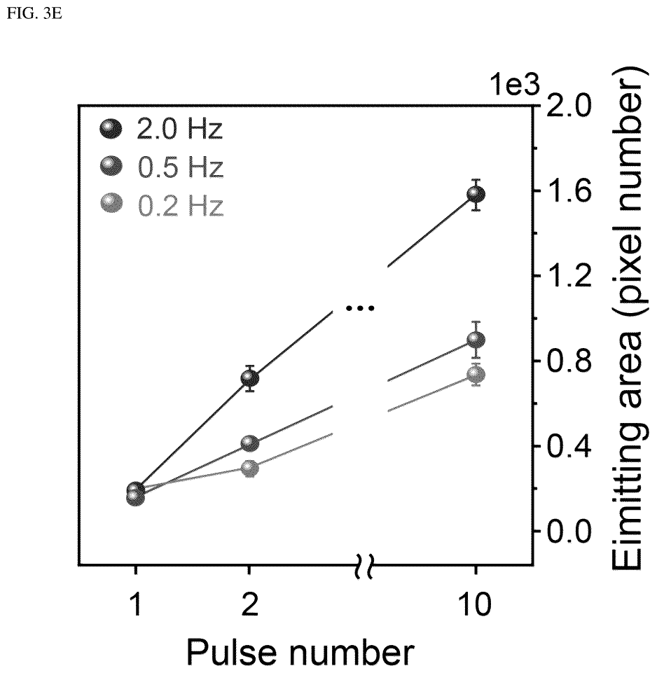

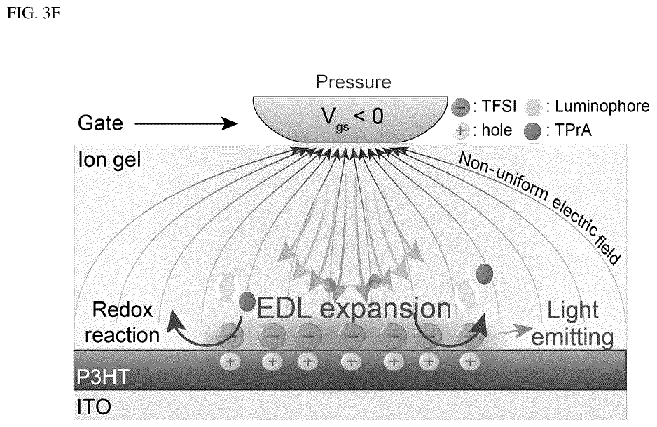

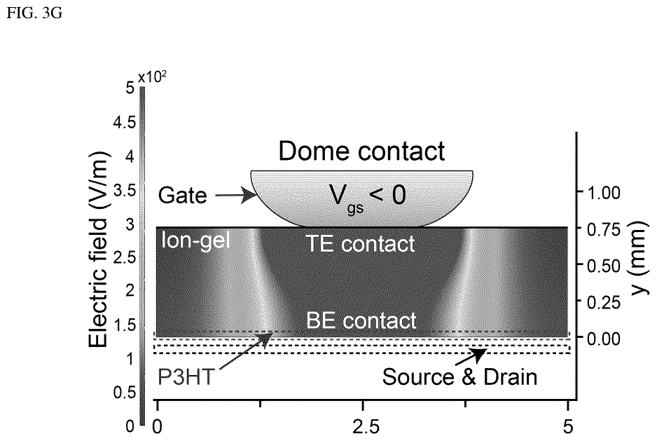

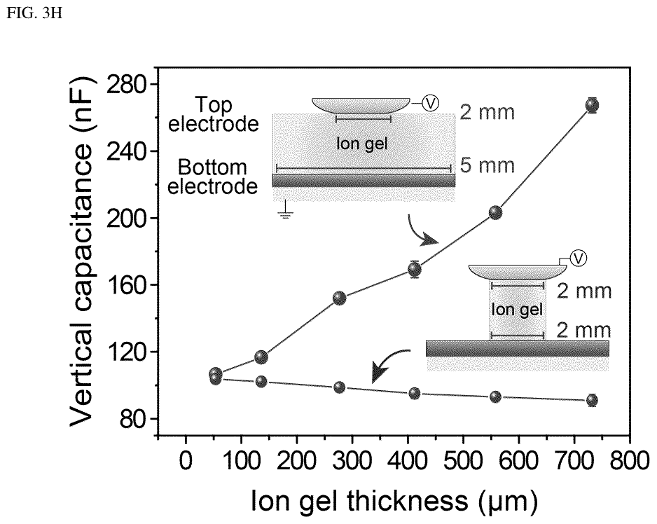

Based on the previously described working mechanism, the tactile synaptic activity represented by the source-drain current in the P3HT channel was successfully visualized in ECL in an R-electrochemiluminescent tactile visual synaptic element (ECL-TVS), and the results are shown in FIG. 3A and FIG. 3B. Specifically, constant negative gate voltage of −3.5 V were applied to the electrochemiluminescent tactile visual synaptic element (ECL-TVS), sufficiently large for the oxidation of the Ru(bpy)₃²⁺ and TPrA, which in turn triggered the ECL reaction. The level of PSC and ECL increased with the number of applied gate tactile pulses, as shown in FIG. 3A. The PSC exhibits a dynamic decay and remains present even after the pulse is applied, whereas the ECL emits light only during pulse application and completely turns off afterwards. Interestingly, the tactile synaptic activity induced by the tactile pulses was visualized as red ECL, with both the intensity and area of ECL increasing concurrently with the number of pulses, as shown in the series of photographs and plot in FIG. 3B, which illustrates the unique feature of the bioinspired full-color electrochemiluminescent tactile visual synaptic element (ECL-TVS).

The tactile pulse frequency-dependent synaptic plasticity of the bioinspired full-color electrochemiluminescent tactile visual synaptic element (ECL-TVS) was also examined, and the results are shown in FIG. 3C. The PSC level increased based on the gate tactile frequency in the order of 0.1 Hz, 0.5 Hz, and 2.0 Hz, which is consistent with the results of the electrical pulse frequency-dependent synaptic plasticity of the bioinspired full-color electrochemiluminescent tactile visual synaptic element (ECL-TVS). Both intensity and light emitting area are further enhanced with the pulse frequency. The light intensity PPF (I-PPF) and emitting area PPF (A-PPF) are plotted as a function of the tactile pulse frequency, as shown in FIG. 3D and FIG. 3E, respectively. These light-emitting synaptic results qualitatively resemble the bioluminescent behavior observed in Hinea brasiliana. The ECL-TVS of this study exhibits an increase in the light emitting area with the number and frequency of the tactile pulse (FIG. 3B and FIG. 3E). These unique results in the light emitting area arose from the gate electric field-dependent ECL reaction in a thick volumetric ion-gel with a thickness of approximately 750 μm, as schematically illustrated in FIG. 3F. Following consecutive gate pulses, the area of ECL was controlled by the asymmetric electric field between the top gate and bottom drain electrode developed by the geometric design of both electrodes and an ion-gel. Numerical simulations of the electric field distribution in the present ECL-TVS were conducted using finite element analysis (FEA), as depicted in FIG. 3G.

In particular, in the bioinspired full-color electrochemiluminescent tactile visual synaptic element (ECL-TVS) developed in the present disclosure, the area between the P3HT and ion-gel is larger (5 mm) than that of the gate electrode (2 mm), and the capacitance of the bioinspired full-color electrochemiluminescent tactile visual synaptic element (ECL-TVS) was significantly increased with ion-gel thickness (FIG. 3H). In this ‘asymmetric geometry capacitor’ situation, the light emission area was readily controlled by varying the relative dimensions of the two electrodes and the thickness of an ion-gel, as shown in FIG. 3F and FIG. 3G. Consequently, owing to the asymmetric area capacitance effect, sufficient ECL reactions are observed at the center and in its vicinity with gate pulses, increasing the light-emitting area with pulses. To confirm this speculation, the inventors of the present disclosure prepared the electrochemiluminescent tactile visual synaptic element (ECL-TVS) using an ion-gel with a width of 2 mm (FIG. 3H). As expected, the capacitance of an ion-gel was rarely altered as a function of the film thickness because the capacitance of an ion-gel was governed by an electrical double layer (EDL), approximately 1 nm, formed at the interface, which ensures the area of ECL rarely changes. The emission area increase is caused by the ‘asymmetric geometry capacitor effect’.

### [Full-Color Electrochemiluminescent Tactile Visual Synaptic Element (ECL-TVS)]

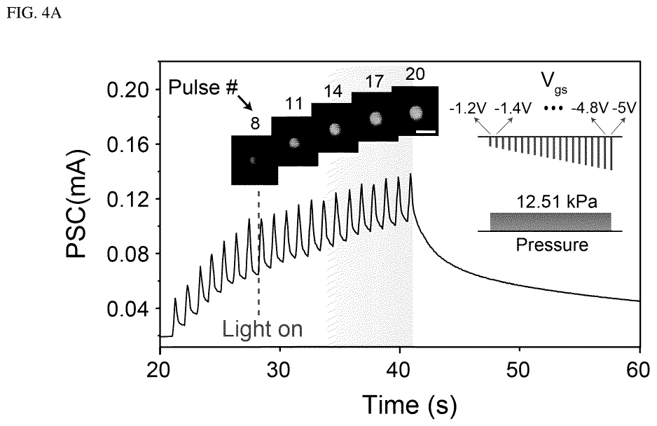

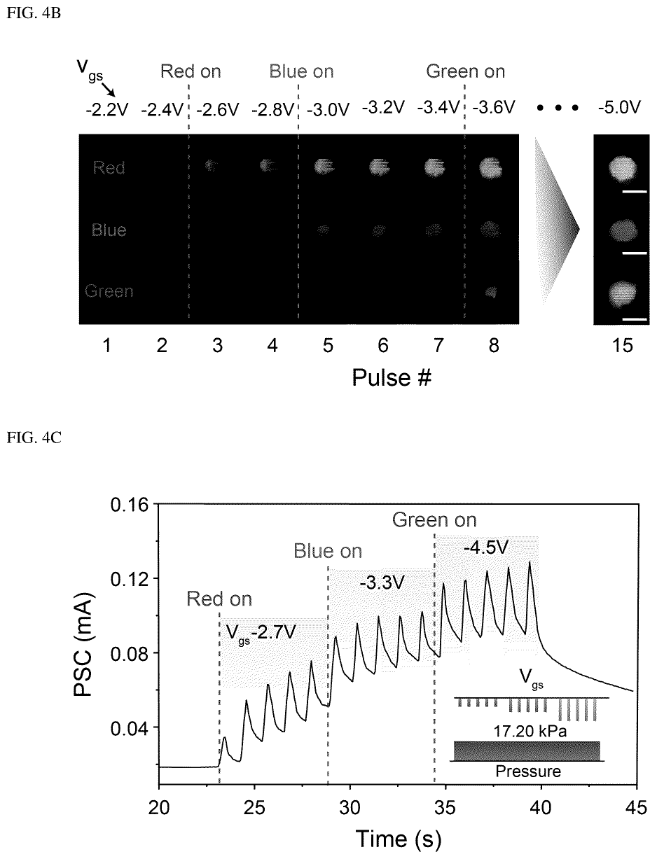

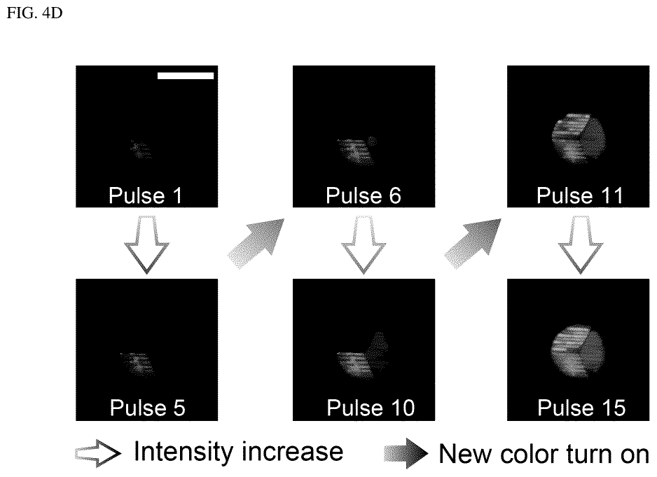

The electrochemiluminescent tactile visual synaptic elements (ECL-TVS) enabling the visualization of the tactile synaptic activity with a fully visible range ECL was developed, and the results are shown in FIG. 4. As previously demonstrated in FIG. 1F, the proposed ECL-TVS confirms that the turn-on behavior of ECL may be controlled through the function of the gate voltage, owing to the specific oxidation potential of the luminophores. By employing the luminophores with different oxidation potentials, combined with the control of the gate voltage of the hemispherical gate, the inventors of the present disclosure could develop the electrochemiluminescent tactile visual synaptic elements (ECL-TVS) where the tactile synaptic activity was visualized in various ECLs with different energies. Before the development of a full-color electrochemiluminescent tactile visual synaptic element (ECL-TVS), the inventors of the present disclosure investigated the gate-voltage dependent ECL turn-on of the electrochemiluminescent tactile visual synaptic elements (ECL-TVS) with the R-luminophore gradually varying the amplitude of the electric pulses from −1.2 V to −5.0 V by 0.2 V for the 20 electric pulses, and the results are shown in FIG. 4A. As depicted in FIG. 4A, while the electrical synaptic potentiation occurred immediately with the electric pulses imposed on the bioinspired full-color electrochemiluminescent tactile visual synaptic element (ECL-TVS), the characteristic red ECL was turned on at the 8th pulse, which corresponded to the gate voltage of −2.6 V, slightly higher than the turn-on voltage of the R-luminophore. Subsequently, both ECL intensity and area increased with the electric pulses. Notably, the overpotential field imposed on the P3HT arising from the gradual increase in gate voltage may further enhance the ECL intensity and area.

By utilizing the characteristic oxidation voltage-dependent ECL turn-on in the present electrochemiluminescent tactile visual synaptic element (ECL-TVS), the inventors of the present disclosure successfully demonstrated red, green, and blue ECL, as shown in FIG. 4B. As observed in FIG. 4B, the red, blue, and green ECL were turned on at the gate pulses of −2.6 V (3rd pulse), −3 V (5th pulse), and −3.6 V (8th pulse), respectively, consistent with the results shown in FIG. 1F. The gate-voltage dependent turn-on of the R-, G-, and B-luminophore enabled us to develop a full-color electrochemiluminescent tactile visual synaptic element (ECL-TVS), as schematically shown in FIG. 1B. In the full-color electrochemiluminescent tactile visual synaptic element (ECL-TVS), three pieces of the ion-gels containing R-, G-, and B-luminophore were cut equally in size and stuck on a P3HT channel layer, creating a combined ion-gel with a diameter of 5 mm.

The inventors of the present disclosure identified the electric synaptic responses of the bioinspired full-color electrochemiluminescent tactile visual synaptic element (ECL-TVS) to 15 electric pulses, applied sequentially at gate voltages of −2.7 V, −3.3 V, and −4.5 V, as shown in FIG. 4C. The red ECL was activated at the first pulse and intensified over the next four pulses, as shown in the left photographs of FIG. 4D (pulse 1 to 5). At the 6th pulse (−3.3 V), the blue ECL turned on and intensified with successive pulses, while the red ECL also strengthened, as shown in the middle photographs of FIG. 4D (pulse 6 to 10). By the 11th pulse (−4.5 V), the green ECL activated, leading to full-color operation as all ECL intensities increased, as shown in the right photographs of FIG. 4D (pulse 11 to 15). The final pulses (12th to 15th) further enhanced the red, green, and blue ECLs (see Supplementary Video 1). The development of an ion-gel containing [PYR₁₄][TFSI] with a wide electrochemical stability window (up to −5 V) was crucial for achieving full-color operation by controlling the oxidation potentials of R-, G-, and B-luminophores. This tactile sensing full-color electrochemiluminescent tactile visual synaptic element (ECL-TVS) holds significant potential for health monitoring applications.

[Personalized Finger Motion Monitoring and Rehabilitation with a Wearable ECL-TVS]

Finger rehabilitation aims to restore delicate and smooth movement of finger joints essential to touching and grasping objects, directly related to physical recognition. The rehabilitation results from the time-consuming incremental function restoration often require the patient to perform repetitive, long-term finger exercises under expert guidance according to proper medical protocols. Such finger function recovery strongly depends upon the strength and number of finger exercises and the frequency of the exercise. Despite its importance, finger rehabilitation often faces challenges such as a lack of providers, limited access to assistive technology, and a scarcity of specialized rehabilitation services. Although several assisting elements for finger rehabilitation are commercially available, most of them are physically bulky and mechanically assembled with various electrical components, which typically target patients who are unable to undergo rehabilitation independently. The inventors of the present disclosure envisioned that the present single element, thin film type electrochemiluminescent tactile visual synaptic element (ECL-TVS), could be suitable as a tailor-made lightweight wearable medical element for finger rehabilitation, aimed at those who may exercise independently but require consistent rehabilitation support. More importantly, the ECL visualization of the tactile synaptic activity on the electrochemiluminescent tactile visual synaptic elements (ECL-TVS) offers convenient visual guidance to the patients by informing them of the progress in long, repetitive, and time-consuming exercises.

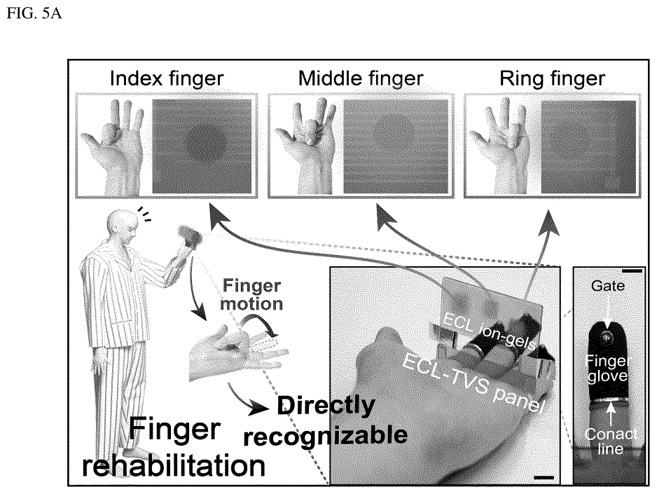

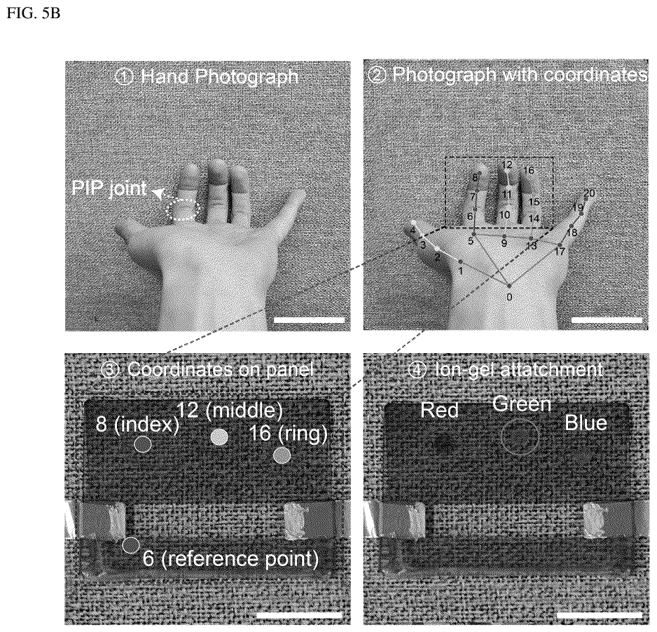

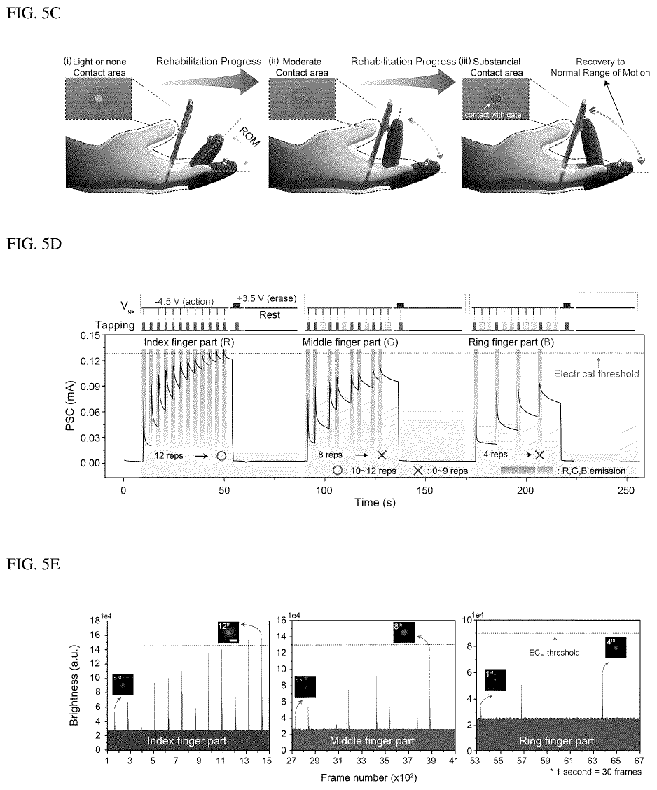

The inventors of the present disclosure developed a personalized wearable element to help with the rehabilitation of various finger motions to demonstrate the finger rehabilitation facilitated with an electrochemiluminescent tactile visual synaptic element (ECL-TVS). Among these, the inventors of the present disclosure emphasized the functional rehabilitation of finger bending, a crucial function in daily life for actions such as writing, typing, and grasping. As shown in FIG. 5A, electrochemiluminescent tactile visual synaptic elements (ECL-TVS) were fabricated with red, green, and blue ECL chromophores on a glass substrate, followed by being vertically fixated on a palm. Three elastomeric finger caps with hemispherical gold deposited PDMS top gates were worn on the index, middle, and ring fingers, respectively, as shown in FIG. 5A. The inventors of the present disclosure set up the activity visualization's red, green, and blue ECL for the index, middle, and ring finger bending motions, respectively. A 7.5 cm×5 cm sized panel element with the three electrochemiluminescent tactile visual synaptic elements (ECL-TVS) was successfully developed and firmly mounted on a palm, as shown in the left inset of FIG. 5A. A finger cap composed of a lightweight and easy-to-wear commercial rubber film was also successfully developed with an elastomeric hemispherical top, as displayed in the photograph in the inset of FIG. 5A. A 2 mm diameter hole was made with masking tape in each ECL-TVS to precisely control the area of tactile touch during the finger exercise, as shown in the right insets of FIG. 5A.

The locations of the three electrochemiluminescent tactile visual synaptic elements (ECL-TVS) on a finger rehabilitation panel, allowing optimal contact with the three fingers, were patient-dependent. They were determined based on a personalized hand recognition algorithm combined with image analysis, and the results are shown in FIG. 5B. In brief, the finger joints shown in the photographs were numbered using the personalized hand-recognition algorithm. The inventors of the present disclosure used the proximal interphalangeal (PIP) (No. 6) of the index finger as a reference point to obtain the coordinates of the fingertips for the index, middle, and ring fingers of a patient. As a final step, red, green, and blue ion-gels, 5 mm in diameter, were carefully placed on the obtained coordinates, as shown in FIG. 5B. The personalized finger position recognition coding was also performed with 16 individuals, providing reliable and consistent ECL in the developed wearable finger rehabilitation panel. The proper location of the electrochemiluminescent tactile visual synaptic elements (ECL-TVS) on the panel using the present personalized finger recognition coding was identified by reliable ECL emission upon finger contact events, as shown in the series of photographs.

The degree of finger rehabilitation is related to an enhancement in the ‘Range of Motion’ (ROM) of the target joint's movement. The American Medical Association (AMA) standard stipulates a normal proximal interphalangeal (PIP) joint ROM 100 degrees which plays a central role in finger function and has significant involvement in the total finger range of motion. As illustrated in FIG. 5C, a user with an initially low ROM value could not tap the electrochemiluminescent tactile visual synaptic elements (ECL-TVS) panel on his palm owing to the lack of PIP capability. The ROM would be increased by performing the finger exercise described previously, and at a certain ROM, the finger touches the panel, resulting in both electrical and ECL output from the bioinspired full-color electrochemiluminescent tactile visual synaptic element (ECL-TVS). Following that, the enhancement in ROM is directly correlated with the increase in the contact area of a finger on the bioinspired full-color electrochemiluminescent tactile visual synaptic element (ECL-TVS) panel. The improved finger movement with the enhanced ROM facilitated firm contact of the finger on the panel with the increased contact area, increasing the electrical and ECL output. Moreover, at a given threshold electrical current and ECL output value, the number of finger touch events required to satisfy the specific threshold values would decrease as the finger contact areas increase. At a certain electrical and ECL output resulting from the finger rehabilitation exercise prescribed from the protocol, the bioinspired full-color electrochemiluminescent tactile visual synaptic element (ECL-TVS) lets a patient know that the exercise was satisfactory.

Before employing a wearable ECL-TVS on a group of participants whose ROM values were lower than 90 degrees (lower than the AMA standard), an exemplary test, including a rehabilitation exercise (finger flexion) program, was performed to verify the bioinspired full-color electrochemiluminescent tactile visual synaptic element (ECL-TVS)'s usefulness. The exercise began with the delivery of 12 gate pulses at −4.5 V, exceeding the electrochemical potentials of the red, green, and blue luminophores used in the procedure.

The inventors of the present disclosure examined bending of the index, middle, and ring fingers. The electrical threshold of 0.13 mA was consistent across all fingers, with ECL thresholds set at 1.45×10⁶ Σbrightness for the index finger, 1.3×10⁶ Σbrightness for the middle finger, and 9×10⁵ Σbrightness for the ring finger, achievable with ten moderate taps (12.51 kPa). As shown in FIG. 5D and FIG. 5E, the electrical and ECL output data were collected when the index, middle, and ring fingers were successfully tapped on the wearable panel for 12, 8, and 4 taps, respectively. Both electrical and ECL were intensified with the taps, as shown in FIG. 5D and FIG. 5E, respectively. The brightness extraction algorithm was developed to quantify the ECL level. As expected, only the index finger, with its 12 successful taps via PIP motions, exceeded both electrical (0.13 mA) and ECL threshold values (1.45×106 Σbrightness), which are indicated by the dotted lines shown in FIG. 5D and FIG. 5E. Eight and four taps of the middle and ring finger bending motions on the panel also resulted in both electrical and ECL emissions; however, the extent of the electrical and ECL emissions were not sufficiently large to reach the threshold values. When the exercise was performed adequately with all three fingers, both electrical and ECL results successfully exceeded the threshold values. The method successfully tracked the rehabilitation progress and provided visualized feedback to participants immediately.

Notably, the developed wearable rehabilitation element consumes power only when a finger exercise is performed with 12 discrete events (e.g., finger bending motions). The gate voltage was applied in pulses at each bending event. Based on the 12 consecutive cycles shown in FIG. 5D, the total energy consumption of one person for a 5-week rehabilitation period is 84 mJ. Considering that a commercial coin cell battery with 70 mAh and 1.5 V provides approximately 378 J of energy, the present ECL-TVS may be used for finger rehabilitation of approximately 4500 participants with a single battery. An experiment was conducted to assess the feasibility of utilizing ECL-TVS as a wearable element capable of operating with portable batteries. The successful light-emitting operation of the bioinspired full-color electrochemiluminescent tactile visual synaptic element (ECL-TVS) was powered by four standard batteries. Furthermore, the inventors of the present disclosure demonstrate the long-term performance of pulse-based ECL-TVS as a wearable element for finger rehabilitation, utilizing ion-gel replacement. The results of this study demonstrate the effectiveness of the wearable ECL-TVS element in providing self-monitoring finger motion by enabling participants to track their own electrical and ECL output. The findings highlight the potential of the bioinspired full-color electrochemiluminescent tactile visual synaptic element (ECL-TVS) panel as an effective tool for enhancing finger rehabilitation outcomes, providing personalized and effective therapy, and fostering patient involvement in recovery. Additionally, the potential application of the bioinspired full-color electrochemiluminescent tactile visual synaptic element (ECL-TVS) in artificial muscle technology is demonstrated through an actuator operation experiment.

[Arrhythmia Monitoring System with Electrochemiluminescent Tactile Visual Synaptic Element (ECL-TVS)]

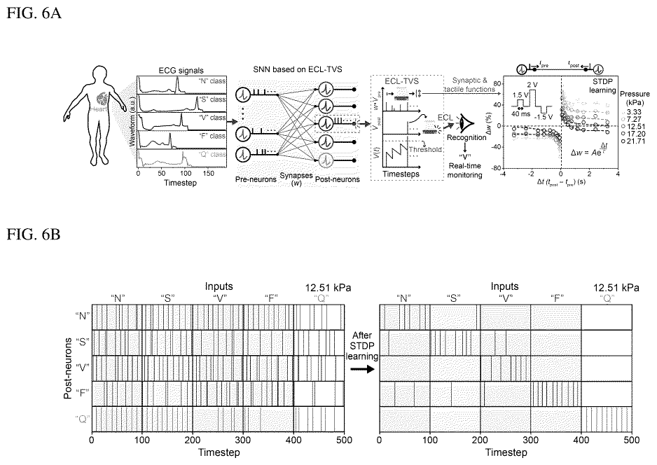

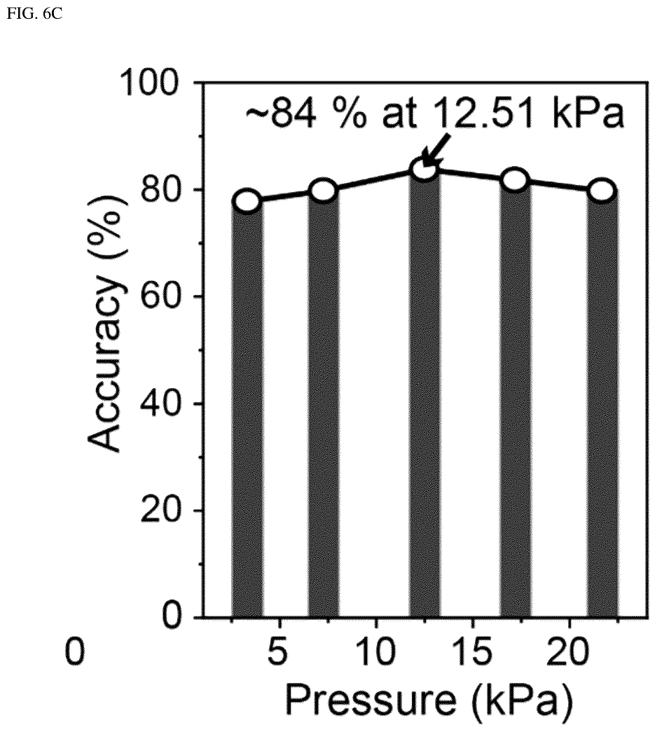

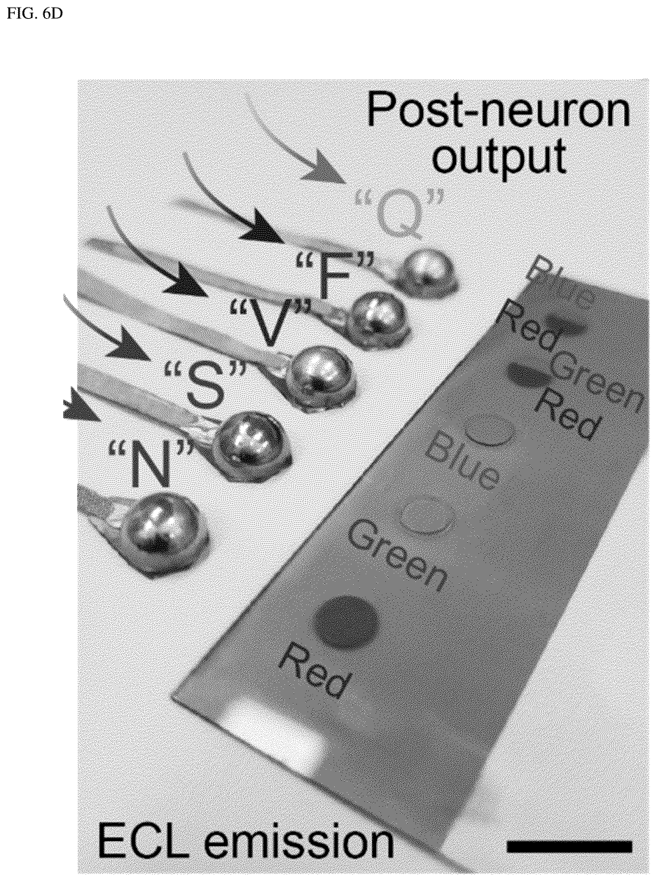

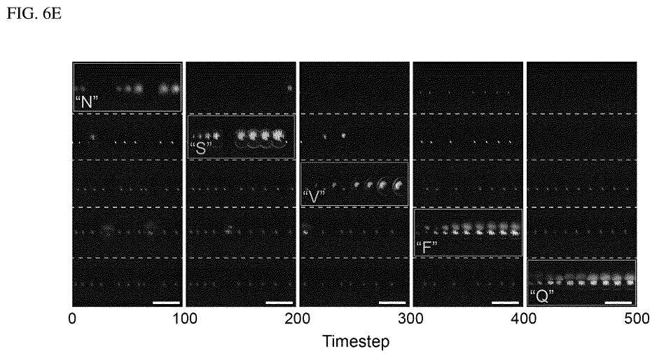

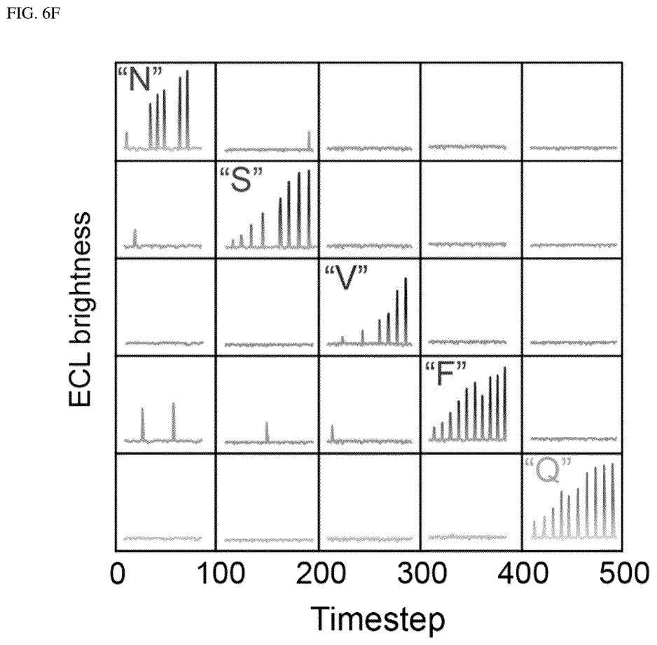

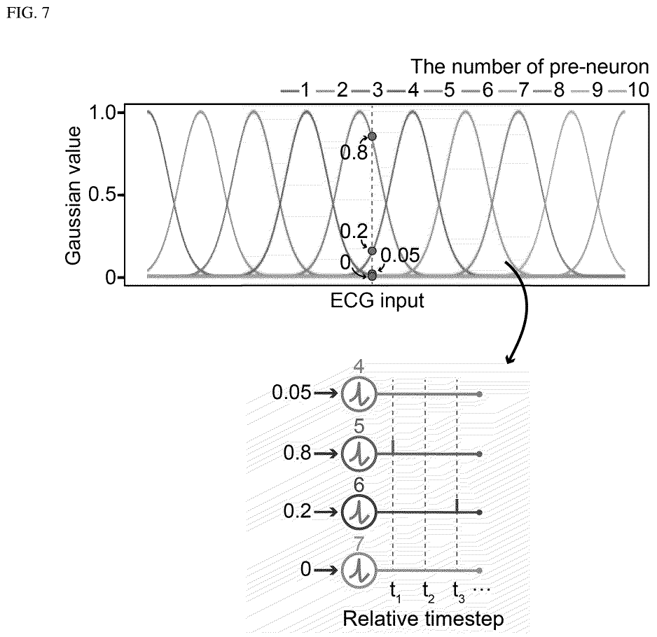

Finally, the inventors of the present disclosure also demonstrate that the present full-color electrochemiluminescent tactile visual synaptic element (ECL-TVS) is suitable for monitoring and directly visualizing various types of arrhythmias with different ECL colors, combined with a spiking neural network (SNN), and the results are shown in FIG. 6A to FIG. 6F. Generally, the heart condition and its electrocardiogram (ECG) are manually interpreted and analyzed by specialists' trained eyes, which is time-consuming and inconvenient for patients. Delays in diagnosing the heart condition could result in patient anxiety and serious irregular heartbeats with high mortality, degrading the overall quality of life. Thus, it is necessary to develop a ubiquitous and user-friendly healthcare system that may readily and promptly interpret heartbeats and ascertain the health status without expertise in the cardiovascular system. Immediate visualization of the heart condition or health status could be an alternative and practical approach for this intelligent health care monitoring system.

The MIT-BIH arrhythmia dataset was employed (in the left diagram of FIG. 6A) to perform the prompt visualization of arrhythmia status. The dataset contains ECG signals corresponding to either normal or abnormal heartbeats. As shown in the left diagram of FIG. 6A, the ECG signals exhibit unique shapes and features of heartbeats over time, which may be labelled into five representative classes such as “N”, “S”, “V”, “F”, and “Q”. The “N” class indicates a healthy state, whereas the remaining classes represent types of arrhythmias. The middle diagram of FIG. 6A shows a diagnostic flow example of SNN based on ECL-TVS with respect to “V”. The SNN, which closely emulates the operating principle of the human brain, operates based on the sequence and timing of spikes, thereby facilitating time-series information processing, such as an ECG signal as compared to conventional artificial neural networks (ANNs). Additionally, since SNN features event-driven signal propagation and local learning, it does not require extensive matrix calculations, thus enhancing energy efficiency compared with ANNs. To encode the ECG signal into a spike, a uniformly distributed population of Gaussian pre-neurons is used. Different ECG signal values produce different excitatory intensities within this population of pre-neurons, which is expressed in the form of a Gaussian curve. For example, strong excitation (pre-neuron 5, purple) results in a short spike generation time (tpre), and weak excitation (pre-neuron 6, brown) produces a longer tpre. On the other hand, if the excitatory intensity is very low (pre-neuron 4, red, <0.1) or not at all (pre-neuron 7, light purple and pre-neuron 1-3, 8-10), no spike occurs in the corresponding neuron (FIG. 7).

As shown in the middle diagram of FIG. 6A, the suggested SNN largely contains synapses fully connected between pre- and post-neurons. In this regard, the inventors of the present disclosure applied ECL-TVS as both synapses and post-neurons based on its synaptic, tactile, and ECL functionalities. When an ECG signal enters the network, pre-neurons transform their temporal inputs into sparse electrical spike trains (Vpre). The post-neurons collect and integrate the incoming spikes from pre-neurons, which are scaled by the strength of the synaptic connection (i.e., synaptic weight, w). If the sum of the weighted spikes (w-Vpre) over time at a certain post-neuron (V(t)) exceeds a threshold, the post-neuron fires an output spike (Vpost) and then visualizes the diagnostic results with ECL.

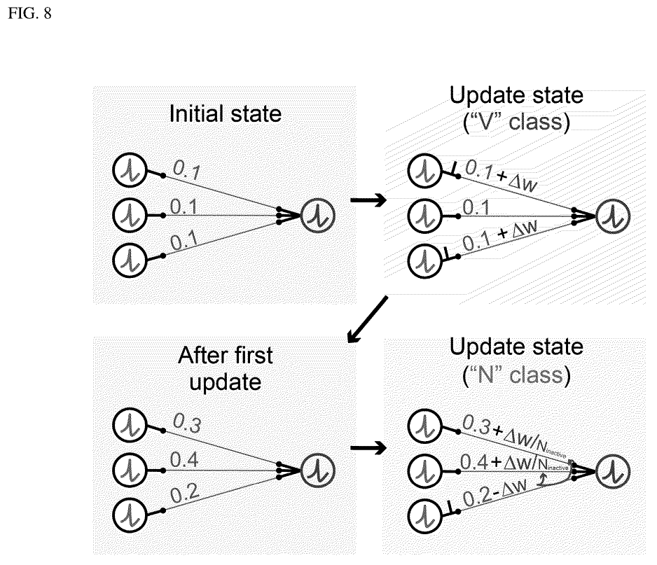

The right diagram of FIG. 6A shows experimental demonstrations of spike-timing-dependent plasticity (STDP) obtained from the electrochemiluminescent tactile visual synaptic elements (ECL-TVS) as a function of different pressures. As shown in the inset of the right diagram of FIG. 6A, STDP, which is a fundamental learning rule of the brain, may determine change in the synaptic weight (Δw) based on the relative spike timing of pre- (tpre) and post-neuron (tpost) (Δt=tpost−tpre). Since the time correlation between tpre and tpost is related to Δw, it is expected that STDP learning may effectively address temporal ECG signals and induce local learning with energy efficiency. Despite the application of the same input voltage scheme (the left inset), the STDP may be further modulated based on the applied pressure. This result indicates that the external pressure inputs enable the in-situ alternation of STDP behavior, which may be utilized as a tuneable learning parameter. All STDP characteristics are well-fitted by the exponential curves and are mathematically expressed as Δw=Δexp(Δt/τ), where A and τ are the fitting parameters. The above process includes an auxiliary step of adjusting the synaptic weight to support STDP learning. The initial synaptic weight is set to 0.1, and when a specific ECG class reaches the post-neuron, the synaptic weight associated with the active pre-neuron increases by Δw. On the other hand, the weight connected to the inactive pre-neuron decreases by Δw/Ninactive, which is to adjust the weight state higher than the initial value (FIG. 8).

FIG. 6B shows firing frequencies of different post-neurons before and after STDP learning over timesteps at 12.51 kPa. Note that “N”, “S”, “V”, “F”, and “Q” are sequentially entered into the network every 100 timesteps (the upper diagram of FIG. 6B), and that the learning was simulated based on the fitting curves. Before learning (the let diagram of FIG. 6B), the post-neurons are observed to irregularly fire regardless of the incoming ECG classes, indicating the complete failure of diagnosis (accuracy, about 12%). However, after the learning (the right diagram of FIG. 6B), each post-neuron fires only when the corresponding ECG class enters the network, exhibiting a considerable improvement in diagnostic accuracy (about 84%) The output spikes are then utilized to implement ECL, allowing the diagnostic results to be readily visualized and identified (the middle diagram of FIG. 6A). FIG. 6C shows a change in the diagnostic accuracy with respect to different pressures. This demonstration suggests that an appropriate tactile input may enhance the learning of the temporal ECG signals by adjusting the STDP behavior. Considering the normal resting blood pressure of an adult ranges from 11 kPa to 16 kPa, the suggested SNN could be potentially extended to on-skin wearable healthcare elements.

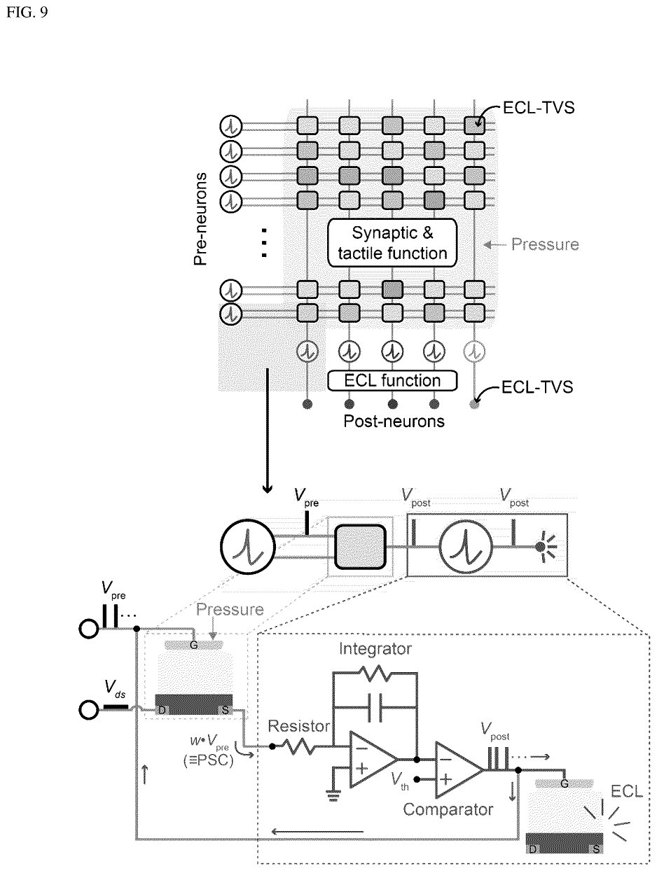

The direct visualization of the trained post-neuron output spikes of “N”, “S”, “V”, “F”, and “Q” (in the right diagram of FIG. 6B) was achieved by five electrochemiluminescent tactile visual synaptic elements (ECL-TVS) with ECL luminophores of red, green, blue, red/green, and red/blue, respectively, as shown in the photograph of FIG. 6D. For the visualization of post-neurons for “F” and “Q” inputs, the inventors of the present disclosure employed an ECL layer containing hemi-circles of red and green and one hemi-circles of red and blue, respectively. The post-neurons mostly corresponding to “N” inputs during the time step from 0 to 100 were visualized in the electrochemiluminescent tactile visual synaptic elements (ECL-TVS) with red luminophores responsible for “N” inputs, as shown in FIG. 6E. As expected, red ECL was intensified with the post-neurons owing to the asymmetric capacitance effect (FIG. 3). In the time step from 100 to 200, the post-neurons that correspond to mostly “S” inputs were visualized in the electrochemiluminescent tactile visual synaptic elements (ECL-TVS) with green ECL (FIG. 6E). Similarly, the post-neurons for “V”, “F”, and “Q” inputs were successfully visualized in the electrochemiluminescent tactile visual synaptic elements (ECL-TVS) with blue, red/green, and red/blue ECL, as shown in FIG. 6E. The intensified ECL with the time-sequential post-neurons of “N”, “S”, “V”, “F”, and “Q” were quantified in FIG. 6F. The ECL-TVS acts as a synapse between a pre-neuron and a post-neuron in SNN, and when an input signal Vpre is input, the weighed spike w·V≡PSC is generated. This spike is transmitted to a circuit composed of a resistor, an integrator and comparator in the post-neuron, which accumulates and compares the input signal. The output signal Vpost generated from the post-neuron is used for synaptic update, and at the same time, suppresses the activity of other post-neurons to induce a “winner-take-all” learning process. In addition, the Vpost is delivered to an additional ECL-TVS to visually represent the ECG diagnosis result (FIG. 9). As shown in the upper diagram of FIG. 9, the proposed SNN for real-time arrhythmia monitoring may be implemented as a crossbar-based circuit in which the ECL-TVS is disposed at each intersection point and the post-neuron terminal. In the STDP learning process, when Vpre is applied to the ECL-TVS synapse (yellow box), the weighted spike (w·V≡PSC) is generated and converted into V(t), which in turn is collected into an LIF post-neuron circuit (green box) composed of a resistor, an integrator and comparator. When V(t) exceeds the set threshold value Vth, Vpost is generated and fed back to the synapse. The synapse is updated according to a temporal correlation (Δt=tpost−tpre) between Vpre and Vpost.

The ECL-TVS may also be adjusted by various external pressures to control the synaptic weight update in real time. In addition, when the post-neuron generates the Vpost, it transmits a suppression signal to prevent other post-neurons from firing during the refractory period. This “winner-take-all” process supports post-neurons to learn ECG signals and effectively distinguish different ECG classes from each other.

Several approaches may be considered to improve diagnostic accuracy. The first approach is to use a larger number of ECG signal data to extract features of the ECG data and facilitate learning. It is identified that despite the use of the small size dataset in FIGS. 6A to 6F, the diagnostic results of the proposed SNN including the ECL-TVS are more accurate compared to the typical ANNs using the larger sized dataset. This result indicates that the SNN is more suitable for temporal information learning and processing.

A second approach is to optimize the encoding scheme. In the pre-neuron encoding scheme, various parameters, such as population size and Gaussian deviation, may affect spike production. Since the number and timing of spikes are related to learning ability, these parameters need to be further optimized. Likewise, the constants (e.g., in, R, and Δtn) of the LIF post-neurons, the auxiliary step C, and the STDP curve operation (e.g., voltage amplitude, width, external pressure) need to be adjusted. It is determined that there is a possibility to further optimize the performance of the SNN by carefully adjusting the device and network parameters.

This demonstration suggests a framework for real-time arrhythmia detection and visualization application with full-color electrochemiluminescent tactile visual synaptic element (ECL-TVS), promising an intelligent healthcare display at the edge.

### [Discussion]

The inventors of the present disclosure demonstrated that the tactile visual synaptic element is potentially suitable for electrically and optically monitoring the movements of the body parts, offering a broad range of healthcare applications, including personalized therapy, intelligent healthcare, and physiological monitoring. The electrochemiluminescent tactile visual synaptic element (ECL-TVS) was developed based on a top-gated OECT, and its emission was conveniently modulated in the full visible range with the gate voltage of the bioinspired full-color electrochemiluminescent tactile visual synaptic element (ECL-TVS). Sensing, learning, and visualizing a variety of tactile events were power-efficiently (about 34 μW) accomplished in the tactile visual synapse. By exploiting the excellent tactile-sensitive synaptic visualization of the proposed element, a lightweight, ultra-low power wearable electrochemiluminescent tactile visual synaptic element (ECL-TVS) was demonstrated with which repetitive bending motions of the finger were monitored in both electrical and optical modes, combined with an output feedback algorithm. Remarkably, the wearable electrochemiluminescent tactile visual synaptic element (ECL-TVS) suited to a palm and a finger promotes the rehabilitation of the finger, enabling efficient tracking of the progress made in the recovery process.

Furthermore, the advanced physiological monitoring with SNN learning of ECG data was demonstrated. The integration of embedded electronics with advanced machine learning algorithms could enable more predictive analytics, offering pre-emptive adjustments to therapy protocols based on patient-specific data trends. This first-of-its-kind, motion monitoring element with visual feedback based on artificial visual synapse, sets a promising foundation, inspiring future research and development in various biomedical applications while exploring new light-emitting materials and element configurations.

### [Methods]

### [Materials]

Processing solvents, acetone, 2-propanol, and toluene were purchased from Sigma-Aldrich. PDMS (Sylgard 184) and its crosslinkers were purchased from Dow Corning. P3HT (Mw=50,000-75,000) with a regioregularity above 90%, P(VDP-HFP) (Mw about 400,000, Mn about 130,000), TPrA, and Ru(bpy)₃Cl₂ (99.95%) were purchased from Sigma-Aldrich. Additionally, 1-butyl-1-methylpyrrolidinium bis(trifluoromethylsulfonyl)imide ([PYR₁₄][TFSI]) and Ir(dFppy)₂(bpy) were purchased from TCI. Firpic was purchased from Ossila. The P3HT solution was prepared by dissolving it in toluene at a concentration of 10 mg mL-1 and being heated on a hot plate for 1 h at 65° C. for the coating of the semiconductor layer. The ECL ion-gel solution was prepared by dissolving P(VDF-HFP) and [PYR₁₄][TFSI] in acetone at a 1:4:10 weight ratio. Subsequently, luminophores and TPrA were added to the solution, maintaining a weight ratio of 0.25 and 2, respectively, relative to the weight of P(VDF-HFP). First, P(VDF-HFP) was dissolved in acetone under continuous stirring at 65° C. for 2 h. Subsequently, [PYR₁₄][TFSI] was added to the solution, and the mixture was stirred at 65° C. for an additional 30 min. Subsequently, luminophores were introduced into the solution, stirring at 60° C. for 6 h. Finally, TPrA was added to the solution, and the temperature was lowered to 50° C. to prevent thermal degradation. The mixture was then stirred for 2 h before use. The entire process was carried out inside a glove box to minimize the humidity contact.

### [Element Fabrication]

A glass substrate was cleaned using an ultrasonic bath, first with acetone and subsequently with 2-propanol, each for 10 min. First, the ITO (60 nm thick) source/drain (S/D) electrodes were sputtered onto the cleaned substrate. Following the sputtering, the deposited ITO was patterned using photolithography to form the source/drain (S/D) electrodes. Subsequently, the P3HT solution was spin-coated on the S/D electrodes, employing a speed of 2000 rpm for 60 s. Heat treatment was administered at 130° C. for 30 min to enhance the P3HT mobility 12. The drop-casted ion-gel was then transferred to the bioinspired full-color electrochemiluminescent tactile visual synaptic element (ECL-TVS). Drop-casting was performed on a hot plate at 60° C. After 5 min, a PET film was floated on the solution to ensure a planar gel surface. Following heat treatment for 1 h and film removal, the remaining solvent was allowed to evaporate in ambient conditions for 12 h. Subsequently, the ion-gel was transferred onto the P3HT using the cut-and-stick method. PDMS (mixed in a base and curing agent ratio at 10:1) was poured onto a dome-shaped Si mold and annealed at 80° C. for 12 h to facilitate hardening to fabricate the elastomeric top gate. Thereafter, 2 min 30 s of 02 plasma treatment was applied to the molded PDMS, and Cr/Au (3 nm/60 nm thick) were thermally evaporated onto the dome-shaped PDMS.

### [Element Characterization]

The cross-sectional morphology and elemental composition of the bioinspired full-color electrochemiluminescent tactile visual synaptic element (ECL-TVS) were analyzed using a field-emission scanning electron microscope (FESEM) (JEOL-7800F) and EDX maps, respectively. Transistor properties and synaptic characteristics measurements were performed using a Keithley 4200 semiconductor characterization system and a semiconductor parameter analyzer (4155C, Keysight) equipped with a pulse generator (81104A, Keysight). Pressure application and measurement were performed by utilizing z-axis pressure equipment, paired with force gauges. The luminance and ECL spectra of the bioinspired full-color electrochemiluminescent tactile visual synaptic elements (ECL-TVS) were obtained using a spectroradiometer (Konica CS 2000). Numerical simulations of the electric field distribution within the bioinspired full-color electrochemiluminescent tactile visual synaptic element (ECL-TVS) were conducted using the finite element analysis (FEA) software COMSOL Multiphysics 5.6 (COMSOL Inc.). The impedance of the bioinspired full-color electrochemiluminescent tactile visual synaptic element (ECL-TVS) IGs was measured via electrochemical impedance spectroscopy, sweeping through a frequency range from 100 Hz to 1 MHz, using a multichannel potentiostat (VMP2, Biologic). Cyclovoltammetry was performed at a scan rate of 25 mV/s using the same multichannel potentiostat. Characterization of the crystallinity of the bioinspired full-color electrochemiluminescent tactile visual synaptic element (ECL-TVS) IGs was accomplished via grazing incidence wide-angle X-ray scattering (GIWAXS), performed at the PLS-II 9A U-SAXS beamline at the Pohang Accelerator Laboratory. The X-ray wavelength was set at 1.1069 Å, while the incident angle was modulated between 0.10° and 0.15°.

### [Electrochemical and Spectroelectrochemical Characterization]

All liquid electrolyte-based electrochemical measurements were performed using a BioLogic SP-200 potentiostat with a standard three-electrode system and acetonitrile solution with 0.1 M [PYR₁₄][TFSI] supporting electrolyte. The P3HT thin films spin-coated on ITO coated glass substrates, Ag/Ag+ wire filled with 0.01 M AgNO₃ and 0.1 M tetrabutylammonium perchlorate (TBAP), and a coiled platinum wire were employed as the working, reference, and counter electrodes, respectively. For spectroelectrochemical measurements, a Keen Innovative Solutions OPTIZEN™ POP UV-vis spectrometer was employed. The beam path passed through an electrolyte-filled quartz glass cuvette containing the polymer/ITO/glass working electrode. Prior, a background spectrum of the cell without external bias was recorded prior to in-situ spectroelectrochemical measurements.

### [Development of Hand Recognition and Video Brightness Extraction Algorithms]

Hand recognition and video brightness extraction algorithms were developed within the Google Colab environment. For the hand recognition algorithm, modifications were done to the source code provided by Google MediaPipe, leveraging its robust framework for hand-tracking applications. The video brightness extraction algorithm involved uploading videos to the Colab environment for efficient video processing and brightness analysis in the cloud.

### [Preparation of Dataset for Arrhythmia Monitoring Task]

MIT-BIH arrhythmia dataset contains ECG recordings from 47 subjects, each independently annotated by two or more cardiologists. Based on the AAMI EC57 standard, the annotations may be categorized into five classes. The MIT-BIH dataset was first pre-processed with a 125 Hz sampling frequency to evaluate the monitoring capability of the suggested SNN. All data were cropped and down-sampled to ensure the same dimension, such that each ECG signal contained 187 data points with normalized amplitudes ranging from 0 to 1 over timesteps.

[Arrhythmia Monitoring Task Based on SNN with Electrochemiluminescent Tactile Visual Synaptic Element (ECL-TVS)]

For arrhythmia monitoring, the inventors of the present disclosure designed a SNN comprising 187 pre-neurons, 935 synapses, and 5 post-neurons. The network initialized all the w to 0.1 and then assigned each class to each post-neuron, respectively. To encode ECG signals into spikes, pre-neurons utilized gaussian neuron model as follows:

$$\text{?} ( x ) = \frac{1}{\text{?}} exp ( - \frac{{( x - \text{?} )}^{2}}{2 \text{?}} ) , \qquad ( 1 )$$

$$where \text{?} = {x}_{min} + ( {x}_{max} - {x}_{min} ) \cdot \frac{\text{?}}{n - 1} , for i = 0 , ... , n - 1. \qquad ( 2 )$$

$$\text{?} \text{indicates text missing or illegible when filed}$$

In this regard, x, n, and σ are input, the number of populations at each pre-neuron, and deviation. In this regard, n and σ were set to be 10 and 0.25. Although the number of pre-neurons is effectively increased by n (in the present case, 187×10=1870), this population encoding scheme may facilitate temporal precision and the neuronal variability problem of temporal coding by spreading each input over multiple pre-neurons. Note that constants n and a may be further optimized. The Gaussian pre-neurons sparsely generated either one or no spikes in response to an input. If a relatively strong input is applied to the pre-neuron, the spike latency required to generate one spike (tpre) becomes shorter. Conversely, a relatively weak input makes tpre longer.

Based on the set of tpre and w, when the weighted spikes were delivered into post-neurons, their membrane potentials (V(t)) over timestep (t) changed according to the leaky-integrate-fire (LIF) neuron model:

$$V ( t + Δ {t}_{n} ) = V ( t ) + \frac{Δ {t}_{n}}{\text{?}} ( - V ( t ) + {RI}_{n} ( t ) ) , \qquad ( 3 )$$

$$\text{?} \text{indicates text missing or illegible when filed}$$

where the constants τn, R, and Δtn were respectively set to be 6, 5, and 0.01, while In(t) represents the weighted input at t. If V(t) at a certain post-neuron exceeds a threshold=3.5, the post-neuron fires an output spike at a timestep (tpost), as shown in the left diagram of FIG. 6D.

For the learning of the SNN, the inventors of the present disclosure employed a two-step learning process: i) the supportive step and ii) the STDP learning step. The supportive step aims to adjust initial w values to a certain set of w to enhance the effect of subsequent STDP learning. For example, a post-neuron receives either its corresponding or non-corresponding ECG class; w values were updated to increase (i.e., reward) or decrease (i.e., penalty) by Δw, respectively, based on tpre:

$$Δ w = C \cdot ( 1 - {t}_{pre} ) , \qquad ( 4 )$$

where the constant C was set to be four. Moreover, the penalty is evenly distributed over inactive synapses connected to non-spiking pre-neurons by Δw/Ninactive, where Ninactive is the number of inactive synapses. Note that inactive synapses that have an initial w value are excluded, and the minimum w was set to the initial w. The STDP learning was then performed based on the Δt=tpost−tpre, mathematically expressed as:

$$Δ w = {A}_{\pm } exp ( \frac{Δ t}{\text{?}} ) , \qquad ( 5 )$$

$$\text{?} \text{indicates text missing or illegible when filed}$$

The w values connected to pre-neurons that generate spikes before their corresponding post-neuron fires are strengthened according to experimental STDP curves, and vice versa. The inventors of the present disclosure used 200 and 50 ECG signals for learning and diagnosis. The diagnostic accuracy may be further improved by learning the network with a larger amount of dataset instead of element optimization and changing the SNN framework.

Although the embodiments of the present disclosure have been described above with reference to the accompanying drawings, the present disclosure may not be limited to the embodiments and may be implemented in various different forms. Those of ordinary skill in the technical field to which the present disclosure belongs will be able to appreciate that the present disclosure may be implemented in other specific forms without changing the technical idea or essential features of the present disclosure. Therefore, it should be understood that the embodiments as described above are not restrictive but illustrative in all respects.

What is claimed is:

1. An electrocardiogram (ECG) pattern classification and visualization device comprising:
a sensor for measuring an electrocardiogram signal of a living body;
an artificial neural network configured to receive the electrocardiogram signal from the sensor as an input and transmit an output via signal propagation; and
a synaptic element configured to receive a signal processed by the artificial neural network therefrom and visualize an electrocardiogram pattern based on the received signal,
wherein the synaptic element includes:
a source electrode;
a drain electrode horizontally spaced apart from the source electrode;
an organic semiconductor layer deposited on both the source electrode and the drain electrode so as to be in contact with both the source electrode and the drain electrode;
an ion-gel layer formed on the organic semiconductor layer; and
a gate electrode formed on the ion-gel layer and formed in a shape in which at least one portion of a lower surface thereof is convex downwardly,
wherein the synaptic element is configured to receive the processed signal from the artificial neural network via the gate electrode,
wherein the ion-gel layer includes:
a polymer matrix;
an ionic liquid dispersed in the polymer matrix and having electrical conductivity;
one or more luminophores dispersed in the polymer matrix and generating electrochemical luminescence in response to electrical stimulation; and
a co-reactant transferring electrons to the luminophores in response to the electrical stimulation.

2. The ECG pattern classification and visualization device of claim 1, wherein the artificial neural network includes a spiking neural network (SNN).

3. The ECG pattern classification and visualization device of claim 1, wherein the organic semiconductor layer includes poly(3-hexylthiophene) (P3HT).

4. The ECG pattern classification and visualization device of claim 1, wherein the gate electrode GE is formed in a partial hemispherical shape protruding downwardly.

5. The ECG pattern classification and visualization device of claim 1, wherein the polymer matrix includes P(VDF-HFP) (poly(vinylidene fluoride-hexafluoropropylene)).

6. The ECG pattern classification and visualization device of claim 1, wherein the ionic liquid includes 1-butyl-1-methylpyrrolidinium bis(trifluoromethylsulfonyl)imide ([PYR₁₄][TFSI]).

7. The ECG pattern classification and visualization device of claim 1, wherein the luminophore includes at least one selected from a group consisting of Ru(bpy)₃Cl₂, Ir(dfppy)₂(bpy)PF₆, and Ir(diFppy)₂(pic) (Firpic).

8. The ECG pattern classification and visualization device of claim 1, wherein the co-reactant may include tripropylamine (TPrA).

9. The ECG pattern classification and visualization device of claim 1, wherein the ECG pattern classification and visualization device is configured to classify the ECG pattern into N” (Normal), “S” (Supraventricular Arrhythmia), “V” (Ventricular Arrhythmia), “F” (Fusion Beat), and “Q” (Unknown or Noisy Beats) patterns, wherein the synaptic element is configured to visualize different electrocardiogram patterns using different colors.

10. A method for diagnosing electrocardiogram, the method comprising analyzing an electrocardiogram of a living body using the electrocardiogram pattern classification and visualization device according to claim 1.
